(19) United States
(12) Patent Application Publication
(10) Pub. No.: US 2023/0272253 A1
(43) Pub. Date: Aug. 31, 2023

(54) ADHESIVE COMPOSITION FOR MAKING WATERPROOF BREATHABLE ARTICLES

(71) Applicant: BOSTIK SA, Colombes (FR)

(72) Inventor: Claire GARNIER, VENETTE (FR)

(21) Appl. No.: 18/005,977

(22) PCT Filed: Jul. 19, 2021
(86) PCT No.: PCT/FR2021/051350

§ 371 Date: Jan. 19, 2023

(30) Foreign Application Priority Data
Jul. 22, 2020 (FR) FR2007707

(51) Int. Cl. C09J 183/06

(52) U.S. Cl. CPC C09J 183/06

(57) ABSTRACT

The invention relates to an adhesive composition comprising at least one silyl polymer comprising at least one hydrolyzable alkoxysilane group; at least one polyvinyl ether compound; and at least one curing catalyst, and also to the use of said composition as an adhesive and also to self-adhesive articles comprising at least one support layer and at least one layer of said composition.

### FIELD OF THE INVENTION

The present invention relates to an adhesive composition, and to the use of said adhesive composition for the preparation of self-adhesive articles.

### TECHNICAL BACKGROUND

A hot-melt pressure-sensitive adhesive (HMPSA) is a composition which confers, on the support which is coated therewith, immediate stickiness at room temperature (often referred to as “tack”). This tack advantageously allows the instantaneous adhesion of this composition to a substrate under the effect of a gentle and brief pressure. HMPSAs are widely used in the manufacture of self-adhesive articles, for instance self-adhesive labels which are attached to articles for purposes of presenting information (barcode, name, price, etc.) and/or for decorative purposes, whether during definitive or temporary adhesive bonding operations. HMPSAs are also used for the manufacture of self-adhesive tapes of varied uses. Besides the transparent adhesive tape widely used in daily life, examples that may be mentioned include: the forming and assembling of cardboard packagings; the protection of surfaces for painting operations, in construction; the fixing and bearing of various elements, such as panels, bricks, protruding objects, in the construction of buildings or edifices; the fixing and bearing of metal, plastic or glass parts, which are flat or which have specific profiles, such as electric cables, plastic films, window panes, metal sheets, inscriptions, logos, parts of seats, dashboards, plastic or textile walls, tubes or pipes for the circulation of fluids, notably in the transportation industry; the adhesive bonding of fitted carpets by double-sided adhesive tapes in the building sector.

Waterproof breathable self-adhesive articles can be used in medical applications such as bandages, dressings, electrodes, plasters, in clothing applications such as breathable clothing to cope with the weather or breathable dry clothing exposed to a humid environment, and in construction applications such as roofing or wall cladding, or leaktight window frame systems that still need to be breathable to ensure that moisture is released into the environment.

In particular, the adhesive materials used, for example, in the medical field, such as medical tapes, wound care dressings and consumer bandages, require a high Moisture Vapor Transmission Rate (MVTR) to allow the moisture generated by the skin or by the wound exudate to escape. A high MVTR of the adhesive product prevents moisture from being trapped under the dressing, which could otherwise cause skin maceration.

Thus, breathable articles must meet certain requirements such as a homogeneous appearance, wind resistance, high permeability to water vapor, a certain elasticity, and also a capacity to adhere to different substrates.

Adhesive compositions suitable for the preparation of self-adhesive articles are already known.

For example, WO 2013/136108 A1 notably relates to an adhesive composition comprising at least one silyl polymer, at least one tackifying resin and at least one catalyst, and to its use for making waterproof breathable articles.

WO 2019/115952 A1 notably relates to a multicomponent adhesive composition comprising a composition A comprising at least one silyl polymer, containing at least one hydrolyzable alkoxysilane group, and at least one tackifying resin; and a composition B comprising at least one catalyst and at least one compound C. Compound C is chosen from a compound C1 having a number average molecular mass ranging from 300 g/mol to 500 000 g/mol and a compound C2 having a vapor pressure at 20° C. greater than or equal to 0.08 kPa.

WO 2019/186014 A1 notably relates to an adhesive composition comprising at least one curable silyl polymer and a catalytic composition comprising a tertiary amine and an organometallic compound.

WO 03/087254 A2 notably relates to a hydrophilic adhesive composition characterized in that it comprises a thermoplastic elastomer of the poly(styrene-olefin-styrene) block copolymer type, a tackifier, a liquid plasticizer, water and an amphiphilic copolymer.

WO 2020/016581 A1 notably relates to a composition comprising polymers comprising silyl groups functionalized with urea and amine groups and a tackifying resin, these compositions being used as hot-melt pressure-sensitive adhesives.

These various waterproof breathable self-adhesive articles, and the adhesive compositions of which they are composed, show satisfactory adhesive properties. However, these articles, and the compositions that comprise them, are not necessarily suitable for medical applications. In particular, these articles, and the compositions that comprise them, such as medical tapes, bandages and dressings, are not necessarily suitable for prolonged application to the skin or a wound. Indeed, prolonged application of these articles can lead to maceration of the skin or the wound, which can generate discomfort for the patient, slow down healing, etc.

There is thus a real need to provide an adhesive composition, which allows the manufacture of self-adhesive articles that are suitable for medical use and which have improved water-vapor permeability, without compromising the good adhesion properties. There is also a real need to provide an adhesive composition, which allows the manufacture of self-adhesive articles—such as medical tapes, bandages or dressings—suitable for prolonged application to the skin or wound. There is also a real need to provide an adhesive composition, which allows the manufacture of self-adhesive articles—such as medical tapes, bandages or dressings—limiting or even preventing maceration of the skin or the wound to which they are applied. There is also a real need to provide an adhesive composition, which enables the manufacture of self-adhesive articles—such as medical tapes, bandages or dressings—facilitating wound healing. There is also a real need to provide an adhesive composition, which allows the manufacture of self-adhesive articles that are suitable for the construction market and which have improved water vapor permeability, without compromising the good adhesion properties.

### SUMMARY OF THE INVENTION

The invention relates firstly to an adhesive composition comprising: at least one silyl polymer comprising at least one hydrolyzable alkoxysilane group;
at least one polyvinyl ether compound; and
at least one curing catalyst.

In certain embodiments, the silyl polymer is chosen from:
a silyl polymer of general formula [Chem 2]

(R⁵O)₃₋ₚ(R⁴)ₚSi—R⁶—O—R²—O—R⁶—Si(R⁴)ₚ(OR⁵)₃₋ₚ
a silyl polymer of general formula

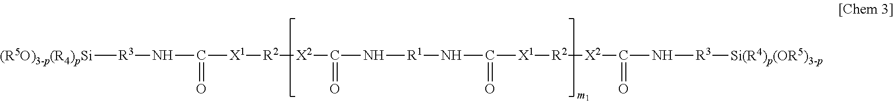

a silyl polymer of general formula

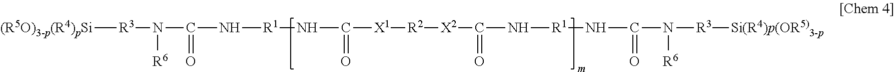

a silyl polymer of general formula

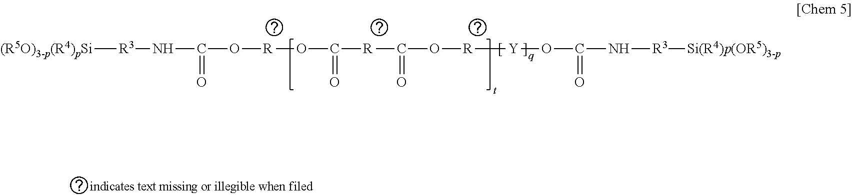

in
which:
X¹ and X² represent, independently of each other, an oxygen atom or an —NH— group;
R¹ represents a divalent hydrocarbon-based radical comprising from 5 to 15 carbon atoms, which may be aromatic or aliphatic, linear, branched or cyclic;
R⁰ represents a linear or branched divalent alkylene radical comprising from 3 to 6 carbon atoms;
R³ represents a linear or branched divalent alkylene radical comprising from 1 to 6 carbon atoms, R³ preferably representing methylene or n-propylene;
R² represents a polyether block —R^{pe}—[OR^{pe}]ₙ— in which R^{pe} represents a linear or branched divalent alkylene radical comprising from 2 to 4 carbon atoms;
R⁴ and R⁵, which may be identical or different, each represent a linear or branched alkyl radical comprising from 1 to 4 carbon atoms;
R⁶ represents a hydrogen atom, a phenyl radical, a linear, branched or cyclic alkyl radical comprising from 1 to 6 carbon atoms, or a 2-succinate radical of formula

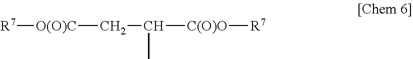

in which R⁷ is a linear or branched alkyl radical comprising from 1 to 6 carbon atoms;
n is an integer such that the number-average molecular mass of the polyether block —[OR^{pe}]ₙ— ranges from 300 g/mol to 40 000 g/mol in the polymers of general formulae [Chem 2], [Chem 3] and [Chem 4];
m₁ is zero or an integer;
m₁ is such that the number-average molecular mass of the polymer of general formula [Chem 3] ranges from 500 g/mol to 50 000 g/mol, preferably from 700 g/mol to 20 000 g/mol;
m is an integer other than zero;
m is such that the number-average molecular mass of the polymer of general formula [Chem 4] ranges from 500 g/mol to 50 000 g/mol, preferably from 700 g/mol to 20 000 g/mol;
p is an integer equal to 0, 1 or 2, p preferably being 0 or 1;
R^{al} represents a divalent hydrocarbon-based radical derived from a diol by replacement of each of the two hydroxyl groups with a free valency or represents the radical R²;
R^{ac} represents a divalent hydrocarbon-based radical derived from a dicarboxylic acid by replacement of each of the two carboxyl groups COOH with a free valency;
[Y]_{q} represents a repeating unit of general formula

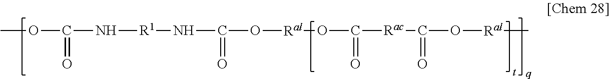

t is a number such that the polyester diol of general formula

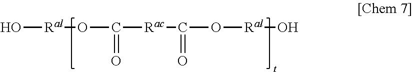

has a hydroxyl number IOH of between 4 and 60 mg KOH/g;
q is an integer other than zero;
t and q are such that the number-average molecular mass of the polymer of general formula [Chem 5] is between 400 g/mol and 50 000 g/mol.

In certain embodiments, the curing catalyst is chosen from the group consisting of amines, organometallic compounds, acids and derivatives thereof, and mixtures thereof.

In certain embodiments, the composition also comprises a tackifying resin; preferably a tackifying resin chosen from terpene phenolic resins, hydrocarbon resins, rosin resins; acrylic resins and mixtures thereof.

In certain embodiments, the polyvinyl ether compound comprises units derived from monomers chosen from methyl vinyl ether, ethyl vinyl ether, butyl vinyl ether, isobutyl vinyl ether, isopropyl vinyl ether, propyl vinyl ether, octyl vinyl ether or combinations thereof.

In certain embodiments, the polyvinyl ether compound is chosen from poly(methyl vinyl ether), poly(ethyl vinyl ether), poly(butyl vinyl ether), poly(isobutyl vinyl ether), poly(isopropyl vinyl ether), poly(propyl vinyl ether), poly(octyl vinyl ether), copolymers thereof and mixtures thereof.

In certain embodiments, the polyvinyl ether compound has a K value of from to 120, and preferably from 40 to 70.

In certain embodiments, the polyvinyl ether compound has a glass transition temperature of from −60 to 0° C., and preferably from −50 to −5° C.

In certain embodiments, the polyvinyl ether compound has a content of from 1% to 60% by weight, preferably from 5% to 40% by weight, and preferably from 5% to 25% by weight relative to the total weight of the composition.

In certain embodiments, the composition also comprises at least one silsesquioxane.

In certain embodiments, said composition is a one-component composition.

In certain embodiments, said composition is a two-component composition comprising:
a part A comprising said at least one silyl polymer; and a part B comprising said at least one curing catalyst;
said at least one polyvinyl ether compound being present in part A and/or in part B of the composition.

The invention also relates to the use of the composition described above as an adhesive.

The invention also relates to a self-adhesive article comprising at least one support layer and at least one layer of the composition described above.

In certain embodiments, said article is chosen from dressings, bandages and medical tapes.

The present invention makes it possible to meet the needs expressed above.

More particularly, it provides a composition for providing a composition that enables the manufacture of articles which have good and improved water vapor permeability, without compromising the good adhesion properties.

This is accomplished by means of the composition according to the invention.

More particularly, the inventors have demonstrated, surprisingly, that the adhesive composition according to the invention—in particular a composition comprising at least one silyl polymer, at least one polyvinyl ether compound and at least one curing catalyst—makes it possible to obtain self-adhesive articles having improved vapor permeability, without compromising good adhesion properties on a substrate. Indeed, the presence of the polyvinyl ether compound makes it possible to obtain good compatibility with the other components of the composition and more particularly with the silyl polymer chains (already having good water vapor permeability), which results in an improvement in the water vapor permeability of the adhesive composition.

The term “good compatibility” means herein stability on storage and long-term stability of the composition.

### DETAILED DESCRIPTION

The invention is now described in greater detail and in a nonlimiting manner in the description that follows.

Adhesive Composition

In a first aspect, the present invention relates to an adhesive composition comprising at least one silyl polymer comprising at least one hydrolyzable alkoxysilane group, at least one polyvinyl ether compound, and at least one curing catalyst. Various additives may also be present in the adhesive composition.

Silyl Polymer

The silyl polymer comprises at least one hydrolyzable alkoxysilane group, preferably as an end group, and preferably at least two hydrolyzable alkoxysilane groups, preferentially as end groups.

The silyl polymer may be a polymer comprising at least one group, preferably at least two groups, of general formula [Chem 1], in particular as end groups:

—Si(R⁴)ₚ(OR⁵)₃₋ₚ
in which:
R⁴ and R⁵, which may be identical or different, each represent a linear or branched alkyl radical comprising from 1 to 4 carbon atoms; and
p is an integer equal to 0, 1 or 2.

When p=2, the groups R⁴ may be identical or different.

When p=1 or p=0, the groups R⁵ may be identical or different.

The silyl polymer comprising at least one, preferably at least two, hydrolyzable alkoxysilane groups may have a main chain chosen from the following main chains: polyether, polyester, polyester-polyether-polyester, polyether-polyester-polyether, polyolefin, polycaprolactone, polyacrylate, polycarbonate, poly(ether-carbonate), poly(ester-carbonate), polyacetal, polyesteramide, polythioether, polyurethane, polyester-polyurethane, polyether-polyurethane, polyether-polyester-polyurethane, polyolefin-polyurethane, polyether-polyolefin-polyurethane, polyurea or poly(urethane-urea).

Preferably, the silyl polymers having at least one hydrolyzable alkoxysilane group are chosen from silyl polyether-polyurethane, silyl polyethers, silyl polyesters, silyl polyester-polyurethane, silyl polyether-polyester-polyurethane, silyl polyureas, silyl poly(urethane-ureas) and mixtures thereof.

The silyl polymer may have a number-average molecular mass (Mn) ranging from 500 to 50 000 g/mol, preferably ranging from 700 to 30 000 g/mol, advantageously from 1000 to 25 000 g/mol, in particular from 1000 to 21 000 g/mol.

The number-average molecular mass of the silyl polymers may be measured by methods that are well known to those skilled in the art, for example by size exclusion chromatography using polystyrene standards.

The silyl polymer including at least one hydrolyzable alkoxysilane group may be chosen from polymers of the general formulae [Chem 2], [Chem 3], [Chem 4] or [Chem 5] as defined below, and mixtures thereof:

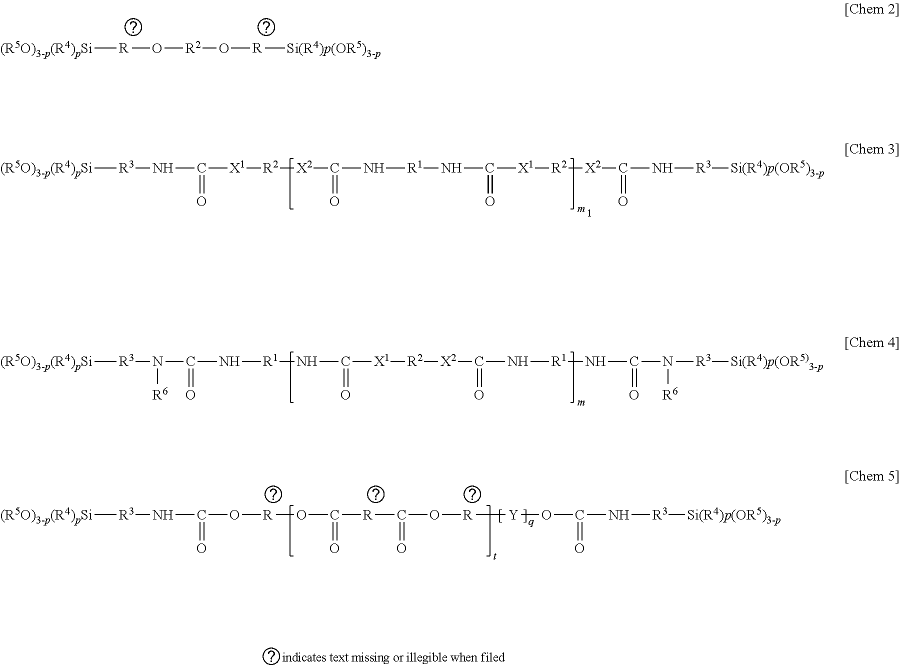

in which:
X¹ and X² represent, independently of each other, an oxygen atom or an —NH— group;
R¹ represents a divalent hydrocarbon-based radical comprising from 5 to 15 carbon atoms, which may be aromatic or aliphatic, linear, branched or cyclic;
R⁰ represents a linear or branched divalent alkylene radical comprising from 3 to 6 carbon atoms;
R³ represents a linear or branched divalent alkylene radical comprising from 1 to 6 carbon atoms, R³ preferably representing methylene or n-propylene;
R² represents a polyether block —R^{pe}—[OR^{pe}]ₙ— in which R^{pe} represents a linear or branched divalent alkylene radical comprising from 2 to 4 carbon atoms;
n is an integer such that the number-average molecular mass of the polyether block —[OR^{pe}]ₙ— ranges from 300 g/mol to 40 000 g/mol in the polymers of general formulae [Chem 2], [Chem 3] and [Chem 4];
R⁴ and R⁵ being as defined above;
R⁶ represents a hydrogen atom, a phenyl radical, a linear, branched or cyclic alkyl radical comprising from 1 to 6 carbon atoms, or a 2-succinate radical of general formula

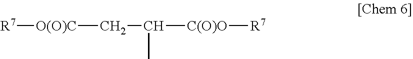

in which R⁷ is a linear or branched alkyl radical comprising from 1 to 6 carbon atoms;
m₁ is zero or an integer;
m₁ is such that the number-average molecular mass of the polymer of general formula [Chem 3] ranges from 500 g/mol to 50 000 g/mol, preferably from 700 g/mol to 20 000 g/mol;
m is an integer other than zero;
m is such that the number-average molecular mass of the polymer of general formula [Chem 4] ranges from 500 g/mol to 50 000 g/mol, preferably from 700 g/mol to 20 000 g/mol;
p is as defined above, p preferably being 0 or 1;
R^{al} represents a divalent hydrocarbon-based radical derived from a diol by replacement of each of the two hydroxyl groups with a free valency or represents the radical R²;
R^{ac} represents a divalent hydrocarbon-based radical derived from a dicarboxylic acid by replacement of each of the two carboxyl groups COOH with a free valency;
[Y]_{q} represents a repeating unit of general formula

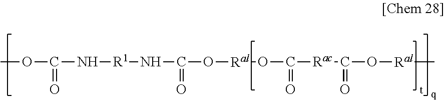

t is a number such that the polyester diol of general formula

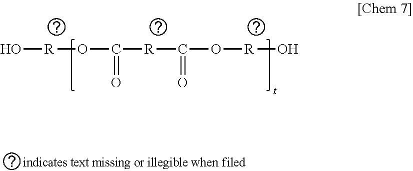

has a hydroxyl number I_{OH} of between 4 and 60 mg KOH/g;
q is an integer other than zero; and
t and q are such that the number-average molecular mass of the polymer of general formula [Chem 5] is between 400 g/mol and 50 000 g/mol.

According to certain embodiments, X¹ and X² are both an oxygen atom. Alternatively, X¹ and X² are both an —NH— group.

Also alternatively, one from among X¹ and X² may be an oxygen atom and the other from among X¹ and X² may be an —NH— group.

Preferably, the radical R¹ of general formulae [Chem 3], [Chem 4] and [Chem 5] is chosen from one of the following divalent radicals, the formulae of which below show the two free valencies:
the divalent radical derived from isophorone diisocyanate (IPDI)

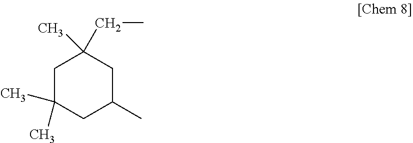

the divalent radical derived from 4,4′- and 2,4′-dicyclohexylmethane diisocyanate (HMDI) of formula

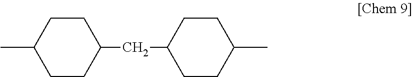

or of formula

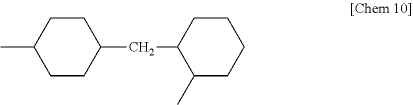

the radical derived from 2,4- and 2,6-toluene diisocyanate (TDI) of formula

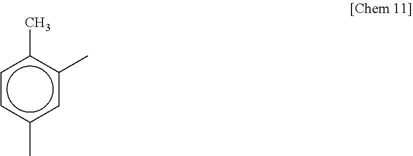

or of formula

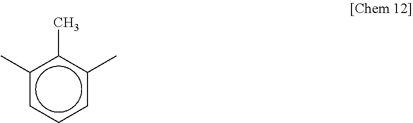

the radical derived from 4,4′- and 2,4′-diphenylmethane diisocyanate (MDI) of formula

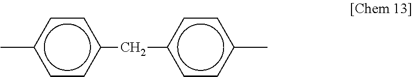

or of formula

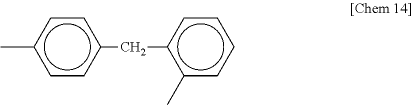

the radical derived from m-xylylene diisocyanate (m-XDI) of formula

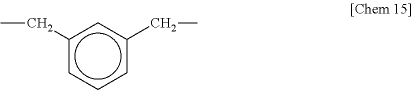

the radical derived from hexamethylene diisocyanate (HDI) of formula [Chem 16]

—(CH₂)₆—

Preferably, the radical R¹ of general formulae [Chem 3], [Chem 4] and [Chem 5] is the divalent radical derived from isophorone diisocyanate or from xylylene diisocyanate.

The polymers of general formula [Chem 3] may be obtained, for example, according to a process described in EP 2336208 A1 and WO 2009/106699 A2.

Among the polymers corresponding to the general formula [Chem 3], examples that may be mentioned include:
Geniosil® STP-E10 (available from Wacker): polyether comprising two groups [Chem 1] of dimethoxy type (m₁ equal to 0, p equal to 1 and R⁴ and R⁵ represent a methyl group) having a number-average molecular mass of about 8889 g/mol where R³ represents a methylene group;
Geniosil® STP-E30 (available from Wacker): polyether comprising two groups of general formula [Chem 1] of dimethoxy type (m₁ equal to 0, p equal to 1 and R⁴ and R⁵ represent a methyl group) having a number-average molecular mass of about 14 493 g/mol where R³ represents a methylene group;
Spur+® 1050MM (available from Momentive): polyether-polyurethane comprising two groups of general formula [Chem 1] of trimethoxy type (m₁ other than 0, p equal to 0 and R⁵ represents a methyl group) having a number-average molecular mass of about 16 393 g/mol where R³ represents an n-propylene group;
Spur+® Y-19116 (available from Momentive): polyether-polyurethane comprising two groups of general formula [Chem 1] of trimethoxy type (m₁ other than 0 and R⁵ represents a methyl group) having a number-average molecular mass ranging from 15 000 to 17 000 g/mol g/mol where R³ represents an n-propylene group;
Desmoseal® S XP 2636 (available from Bayer): polyether comprising two groups of general formula [Chem 1] of trimethoxy type (m₁ equal to 0, p equal to 0 and R⁵ represents a methyl group) having a number-average molecular mass of about 15 038 g/mol where R³ represents an n-propylene group. The polymers of general formula [Chem 2] may be obtained by hydrosilylation of polyether diallyl ether according to a process described, for example, in EP 1 829 928 A1.

Among the polymers corresponding to the general formula [Chem 2], mention may be made of:
MS Polymer™ S303H (available from Kaneka) corresponding to a polyether comprising two groups of general formula [Chem 1] of dimethoxy type (p equal to 1 and R⁴ represents a methyl group) having a number-average molecular mass of about 22 000 g/mol and a viscosity of 12.5 Pa·s at 23° C.; and
MS Polymer™ S227 (available from Kaneka) corresponding to a polyether comprising two groups of general formula [Chem 1] of dimethoxy type (p equal to 1 and R⁵ and R⁴ each represent a methyl group) having a number-average molecular mass of about 27 000 g/mol and a viscosity of 34 Pa·s at 23° C.

The polymers of general formula [Chem 4] can be obtained according to the following process (in the case where the polymer of general formula [Chem 4] is a silyl polyurethane):
reaction of a polyether polyol having the general formula [Chem 17]

HO—R²—OH
with a stoichiometric excess of diisocyanate of formula NCO—R¹—NCO to form a polyurethane-polyether block bearing at least two —NCO end groups, said block preferably comprising from 1.5% to 1.9% by weight of —NCO groups, and then
reaction between a block obtained in the preceding step with a stoichiometric amount or a slight excess of an α-, β- or γ-aminosilane having the general formula [Chem 18]

(R⁵O)₃₋ₚ(R⁴)ₚSi—R³—NHR⁶

Such a process is described, for example, in WO 2013/136108 A1.

Among the polymers corresponding to the general formula [Chem 4], examples that may be mentioned include:
Spur+1015 LM (available from Momentive) corresponding to a polyether-polyurethane comprising two groups of formula [Chem 1] of trimethoxy type (p equal to 0 and R⁵ represents a methyl group) having a number-average molecular mass of about 25 000 g/mol and a viscosity of 50 Pa·s at 23° C.

The polymers of general formula [Chem 4] may be obtained according to the following process (in the case where the polymer of general formula [Chem 4] is a silyl polyurea):
reaction of a diamine of the general formula [Chem 19]

H₂N—R²—NH₂
with a stoichiometric excess of diisocyanate of formula NCO—R¹—NCO to form a polyurea-polyether block bearing at least two —NCO end groups, said block preferably comprising from 1.5% to 1.9% by weight of —NCO groups, and then
reaction between a block obtained in the preceding step with a stoichiometric amount or a slight deficit of an α-, β- or γ-aminosilane having the general formula [Chem 18]

(R⁵O)₃₋ₚ(R⁴)ₚSi—R³—NHR⁶

Such a process is described, for example, in WO 2020/016 581 A1.

The polymers of general formula [Chem 5] may be those described in patent application EP 2 865 694 A1. They may be prepared according to the process described in said patent application.

The polymer of general formula [Chem 5] may be a first polymer of specific general formula [Chem 5] in which:
R^{al} represents a divalent hydrocarbon-based radical which is derived from a fatty alcohol dimerized by replacing each of the two hydroxyl groups with a free valency, said alcohol having a hydroxyl number I_{OH} of between 200 and 220 mg KOH/g;
R^{ac} represents a divalent hydrocarbon-based radical which is derived from a fatty acid dimerized by replacing each of the two carboxyl groups —COOH with a free valency, said acid having an acid number I_{A} of between 190 and 200 mg KOH/g;
t is preferentially a number such that the polyester diol of general formula

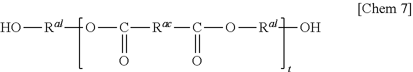

has a hydroxyl number I_{OH} of between 45 and 55 mg KOH/g.

This first polymer of specific general formula [Chem 5] may be obtained according to the process described in patent application EP 2 865 728 A1 (in particular on pages 5 to 9).

Preferably, this first polymer of specific general formula [Chem 5] has a number-average molecular mass ranging from 900 to 15 000 g/mol.

The polymer of general formula [Chem 5] may be a second polymer of specific general formula [Chem 5] in which:
R^{al} represents a divalent hydrocarbon-based radical derived from a saturated diol by replacing each of the two hydroxyl groups with a free valency, said diol having a hydroxyl number I^{OH} of greater than 220 mg KOH/g;
R^{ac} represents a divalent hydrocarbon-based radical derived from a saturated dicarboxylic acid by replacing each of the two carboxyl groups —COOH with a free valency, said acid having an acid number I_{A} of greater than 200 mg KOH/g;
t is preferentially a number such that the polyester diol of general formula

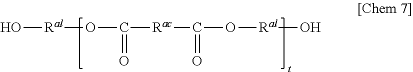

has a hydroxyl number I_{OH} of between 4 and 24 mg KOH/g, in particular between 9 and 24 mg KOH/g.

The second polymer of specific general formula [Chem 5] can be obtained via a process which comprises several sequential steps:

The first step of the process for obtaining the second polymer of specific general formula [Chem 5] consists in preparing a polyester of general formula [Chem 7] having a hydroxyl number I_{OH} of between 4 and 24 mg KOH/g

The polyester of general formula [Chem 7] may be prepared via a polycondensation reaction between:
at least one saturated dicarboxylic acid with an acid number I_{A} of greater than 200 mg KOH/g; and
at least one saturated diol with a hydroxyl number I_{OH} of greater than 220 mg KOH/g.

In the present text:
the acid number I_{A} of a dicarboxylic acid is the number of carboxylic functions per gram of acid, said number being expressed in the form of equivalent in milligrams of KOH necessary to neutralize the acidity of 1 g of acid, determined by titrimetry, said number being related to the number-average molecular mass M of said acid by the following relationship [Math 1]

I_{A}=(56,1×2×1000)/M
the hydroxyl number I_{OH} of a diol is the number of hydroxyl functions per gram of diol, said number being expressed in the form of equivalent in milligrams of KOH used in the assay of the hydroxyl functions, determined by titrimetry according to the standard ISO 14900:2001, said number being related to the number-average molecular mass M′ of said diol by the relationship [Math 2]

I_{OH}=(56,1×2×1000)/M″

Preferably, the saturated dicarboxylic acid(s) have an acid number I_{A} of greater than or equal to 300 mg KOH/g, preferably greater than or equal to 400 mg KOH/g, preferentially greater than or equal to 500 mg KOH/g, in particular greater than or equal to 700 mg KOH/g, and advantageously greater than or equal to 800 mg KOH/g. Preferably, the saturated dicarboxylic acid(s) have an acid number I_{A} equal to 555 mg KOH/g or equal to 768 mg KOH/g.

The dicarboxylic acid may be linear or branched, preferably linear, aliphatic or cycloaliphatic.

The dicarboxylic acid according to the invention may be chosen from the group consisting of malonic acid, succinic acid, fumaric acid, glutaric acid, adipic acid, 1,3- or 1,4-cyclohexanedicarboxylic acid, 3-methyl-1,5-pentanedicarboxylic acid, 1,10-decanedicarboxylic acid, 1,12-dodecanedicarboxylic acid, 1,18-octadecanedicarboxylic acid, methyltetrahydrophthalic acid, hexahydrophthalic acid, tetrahydrophthalic acid, azelaic acid, sebacic acid, and mixtures thereof.

Preferably, the dicarboxylic acid is adipic acid or sebacic acid.

Preferably, the saturated diol(s) have a hydroxyl number I_{OH} of greater than or equal to 500 mg KOH/g, preferably greater than or equal to 700 mg KOH/g, even more preferentially greater than or equal to 900 mg KOH/g.

The diol used may be aromatic or aliphatic (preferably aliphatic), linear or branched, preferably branched.

The diol according to the invention may be chosen from the group consisting of ethylene glycol (CAS: 107-21-1), diethylene glycol, triethylene glycol, tetraethylene glycol, 1,2-propanediol, dipropylene glycol, tripropylene glycol, tetrapropylene glycol, 1,6-hexanediol, 3-ethyl-2-methyl-1,5-pentanediol, 2-ethyl-3-propyl-1,5-pentanediol, 2,4-dimethyl-3-ethyl-1,5-pentanediol, 2-ethyl-4-methyl-3-propyl-1,5-pentanediol, 2,3-diethyl-4-methyl-1,5-pentanediol, 3-ethyl-2,2,4-trimethyl-1,5-pentanediol, 2,2-dimethyl-4-ethyl-3-propyl-1,5-pentanediol, 2-methyl-2-propyl-1,5-pentanediol, 2,4-dimethyl-3-ethyl-2-propyl-1,5-pentanediol, 2,3-dipropyl-4-ethyl-2-methyl-1,5-pentanediol, 2-butyl-2-ethyl-1,5-pentanediol, 2-butyl-2,3-diethyl-4-methyl-1,5-pentanediol, 2-butyl-2,4-diethyl-3-propyl-1,5-pentanediol, 3-butyl-2-propyl-1,5-pentanediol, 2-methyl-1,5-pentanediol (CAS: 42856-62-2), 3-methyl-1,5-pentanediol (MPD, CAS: 4457-71-0), 2,2-dimethyl-1,3-pentanediol (CAS: 2157-31-5), 2,2-dimethyl-1,5-pentanediol (CAS: 3121-82-2), 3,3-dimethyl-1,5-pentanediol (CAS: 53120-74-4), 2,3-dimethyl-1,5-pentanediol (CAS: 81554-20-3), 2,2-dimethyl-1,3-propanediol (neopentyl glycol—NPG, CAS: 126-30-7), 2,2-diethyl-1,3-propanediol (CAS: 115-76-4), 2-methyl-2-propyl-1,3-propanediol (CAS: 78-26-2), 2-butyl-2-ethyl-1,3-propanediol (CAS: 115-84-4), 2-methyl-1,3-propanediol (CAS: 2163-42-0), 2-benzyloxy-1,3-propanediol (CAS: 14690-00-7), 2,2-dibenzyl-1,3-propanediol (CAS: 31952-16-6), 2,2-dibutyl-1,3-propanediol (CAS: 24765-57-9), 2,2-diisobutyl-1,3-propanediol, 2,4-diethyl-1,5-pentanediol, 2-ethyl-1,6-hexanediol (CAS: 15208-19-2), 2,5-dimethyl-1,6-hexanediol (CAS: 49623-11-2), 5-methyl-2-(1-methylethyl)-1,3-hexanediol (CAS: 80220-07-1), 1,4-dimethyl-1,4-butanediol, 1,5-hexanediol (CAS: 928-40-5), 3-methyl-1,6-hexanediol (CAS: 4089-71-8), 3-(tert-butyl)-1,6-hexanediol (CAS: 82111-97-5), 1,3-heptanediol (CAS: 23433-04-7), 1,2-octanediol (CAS: 1117-86-8), 1,3-octanediol (CAS: 23433-05-8), 2,2,7,7-tetramethyl-1,8-octanediol (CAS: 27143-31-3), 2-methyl-1,8-octanediol (CAS: 109359-36-6), 2,6-dimethyl-1,8-octanediol (CAS: 75656-41-6), 1,7-octanediol (CAS: 3207-95-2), 4,4,5,5-tetramethyl-3,6-dioxa-1,8-octanediol (CAS: 76779-60-7), 2,2,8,8-tetramethyl-1,9-nonanediol (CAS: 85018-58-2), 1,2-nonanediol (CAS: 42789-13-9), 2,8-dimethyl-1,9-nonanediol (CAS: 40326-00-9), 1,5-nonanediol (CAS: 13686-96-9), 2,9-dimethyl-2,9-dipropyl-1,10-decanediol (CAS: 85018-64-0), 2,9-dibutyl-2,9-dimethyl-1,10-decanediol (CAS: 85018-65-1), 2,9-dimethyl-2,9-dipropyl-1,10-decanediol (CAS: 85018-64-0), 2,9-diethyl-2,9-dimethyl-1,10-decanediol (CAS: 85018-63-9), 2,2,9,9-tetramethyl-1,10-decanediol (CAS: 35449-36-6), 2-nonyl-1,10-decanediol (CAS: 48074-20-0), 1,9-decanediol (CAS: 128705-94-2), 2,2,6,6,10,10-hexamethyl-4,8-dioxa-1,11-undecanediol (CAS: 112548-49-9), 1-phenyl-1,11-undecanediol (CAS: 109217-58-5), 2-octyl-1,11-undecanediol (CAS: 48074-21-1), 2,10-diethyl-2,10-dimethyl-1,11-undecanediol (CAS: 85018-66-2), 2,2,10,10-tetramethyl-1,11-undecanediol (CAS: 35449-37-7), 1-phenyl-1,11-undecanediol (CAS: 109217-58-5), 1,2-undecanediol (CAS: 13006-29-6), 1,2-dodecanediol (CAS: 1119-87-5), 2,11-dodecanediol (CAS: 33666-71-6), 2,11-diethyl-2,11-dimethyl-1,12-dodecanediol (CAS: 85018-68-4), 2,11-dimethyl-2,11-dipropyl-1,12-dodecanediol (CAS: 85018-69-5), 2,11-dibutyl-2,11-dimethyl-1,12-dodecanediol (CAS: 85018-70-8), 2,2,11,11-tetramethyl-1,12-dodecanediol (CAS: 5658-47-9), 1,11-dodecanediol (CAS: 80158-99-2), 11-methyl-1,7-dodecanediol (CAS: 62870-49-9), 1,4-dodecanediol (CAS: 38146-95-1), 1,3-dodecanediol (CAS: 39516-24-0), 1,10-dodecanediol (CAS: 39516-27-3), 2,11-dimethyl-2,11-dodecanediol (CAS: 22092-59-7), 1,5-dodecanediol (CAS: 20999-41-1), 6,7-dodecanediol (CAS: 91635-53-9), and mixtures thereof.

Preferably, the diol is chosen from the group consisting of ethylene glycol (CAS: 107-21-1), 1,6-hexanediol, 3-methyl-1,5-pentanediol (MPD, CAS: 4457-71-0), 2,2-dimethyl-1,3-propanediol (neopentyl glycol—NPG, CAS: 126-30-7), and mixtures thereof.

Preferably, the polyester of general formula [Chem 7] is obtained by polycondensation reaction between: adipic acid; and a mixture of neopentyl glycol, ethylene glycol, and 1,6-hexanediol;
or between: adipic acid; and 3-methyl-1,5-pentanediol.

Preferably, the polyester diol of general formula [Chem 7] has a hydroxyl number I_{OH} of between 4 and 24 mg KOH/g, preferentially between 7 and 24 mg KOH/g, preferably between 7 and 20 mg KOH/g and in particular between 9 and 19 mg KOH/g. Preferably, the hydroxyl number I_{OH} is between 9 and 24 mg KOH/g.

The polyester diol of general formula [Chem 7] may have a glass transition temperature T_{g} of less than 0° C., preferably less than or equal to −20° C., preferably less than or equal to −40° C., preferentially less than or equal to −50° C., in particular less than or equal to −60° C., for example less than or equal to −64° C.

The polyester diol of general formula [Chem 7] may have a number-average molecular mass of greater than or equal to 5500 g/mol, preferably greater than or equal to 6000 g/mol, in particular strictly greater than 6000 g/mol, preferentially greater than or equal to 8000 g/mol, in particular greater than or equal to 9000 g/mol, for example greater than or equal to 10 000 g/mol, advantageously greater than or equal to 12 000 g/mol and in particular greater than or equal to 18 000 g/mol.

The number-average molecular mass of the polyester diol of general formula [Chem 7] may be determined from its I_{OH} and from its functionality.

Among the amorphous polyester diols of general formula [Chem 7], examples that may be mentioned include Dynacoll® 7250 sold by Evonik (polyester polyol with a viscosity of 180 Pa·s at 23° C., a number-average molecular mass Mn equal to 5500 g/mol, and a T_{g} equal to −50° C.), Kuraray® P-6010 sold by Kuraray (polyester polyol with a viscosity of 68 Pa·s at 23° C., a number-average molecular mass equal to 6000 g/mol, and a T_{g} equal to −64° C.), or Kuraray® P-10010 sold by Kuraray (polyester polyol with a viscosity of 687 Pa·s at 23° C., a number-average molecular mass equal to 10 000 g/mol).

The second step of the process for obtaining the second polymer of specific general formula [Chem 5] consists in preparing the polymer of general formula [Chem 5].

According to a first variant, the abovementioned polyester(s) of general formula [Chem 7] may be reacted with the isocyanatosilane of general formula [Chem 20]

NCO—R³—Si(R⁴)ₚ(OR⁵)₃₋ₚ
in an amount corresponding to a molar equivalent ratio of the number of NCO/OH functions of between 0.90 and 1.05 and preferably equal to about 1.

This step is in particular performed under anhydrous conditions, so as to avoid hydrolysis of the alkoxysilane groups. A typical temperature range for performing this reaction step is from 30° C. to 120° C., and more particularly from 60° C. to 105° C.

The isocyanatosilanes of the abovementioned general formula [Chem 20] are widely commercially available. Mention may be made notably of Silquest® A-Link 35, i.e. (3-isocyanatopropyl)trimethoxysilane) available from Momentive, Silquest® A-Link 25, i.e. (3-isocyanatopropyl)triethoxysilane) available from Momentive, (3-isocyanatopropyl)methyldimethoxysilane available from Gelest, Geniosil® XL 42, i.e. (3-isocyanatomethyl)methyldimethoxysilane available from Wacker, and Geniosil® XL 43, i.e. (3-isocyanatomethyl)trimethoxysilane available from Wacker.

According to a second variant, the silyl polymer may be obtained in two steps by:
reaction of the polyester polyol(s) of general formula [Chem 7] with a diisocyanate of formula NCO—R¹—NCO in amounts corresponding to a molar equivalent ratio of the number of NCO/OH functions of between 0.3 and 0.7 and preferably equal to about 0.5, to form a polyester-polyurethane block;
reaction between a block obtained in the preceding step with the isocyanatosilane of formula [Chem 20], in an amount corresponding to a molar equivalent ratio of the number of NCO/OH functions of between 0.90 and 1.05 and preferably equal to about 1.

The isocyanatosilanes may be those mentioned above.

Preferably, the silyl polymer according to the invention is a polymer of general formula [Chem 3] in which:
m₁ is an integer equal to 0;
p=1;
R⁴ and R⁵ each represent a methyl radical;
R³ represents a divalent methylene radical;
the number-average molar mass of said polymer ranges from 5000 to 30 000 g/mol, preferentially from 10 000 to 20 000 g/mol, in particular from 14 000 to 15 000 g/mol.

Preferably, the silyl polymer according to the invention is a polymer of general formula [Chem 3] in which:
m₁ is an integer other than 0;
p=0;
R³ represents a divalent propylene radical;
R⁵ represents a methyl radical;
the number-average molecular mass of said polymer ranges from 5000 to 30 000 g/mol, preferentially from 10 000 to 30 000 g/mol, in particular from 15 000 to 25 000 g/mol.

Preferably, the silyl polymer according to the invention is a polymer of general formula [Chem 5] in which:
q is an integer other than zero;
p=0;
R³ represents a divalent propylene radical;
R⁵ represents a methyl radical;
the number-average molecular mass of said polymer ranges from 5000 to 30 000 g/mol, preferentially from 10 000 to 30 000 g/mol, in particular from 15 000 to 25 000 g/mol.

The adhesive composition may comprise a single polymer as described above.

Alternatively, the adhesive composition may comprise more than one silyl polymer, for example two, or three, or four, or five, or more than five silyl polymers.

The adhesive composition according to the invention may comprise from 20% to 90%, preferably from 30% to 70% by weight of silyl polymer, relative to the total weight of the adhesive composition.

Polyvinyl Ether Compound

The polyvinyl ether compound according to the invention may be a homopolymer or a copolymer. In the context of the present invention, the term “copolymer” means polymers comprising at least two different monomers. Thus, in the context of the present invention, the term copolymer also includes terpolymers. Preferably, when the polyvinyl ether compound is a copolymer, it is a block copolymer. The term “block copolymer” means a copolymer comprising at least two homopolymer subunits linked together via covalent bonds.

The polyvinyl ether compound may comprise units derived from monomers of general formula [Chem 21]

R—O—C(R⁸)═CH(R⁹)

The group R represents a saturated or unsaturated, linear or branched group comprising from 1 to 24 carbon atoms, preferably from 1 to 10 carbon atoms, preferably from 1 to 8 carbon atoms, and more preferably from 1 to 4 carbon atoms.

The group R may be chosen from the following groups: alkyl, cycloalkyl, aryl, arylalkyl, alkylaryl and a heterocyclic group comprising a ring comprising a heteroatom chosen from oxygen, sulfur and nitrogen; preferably, the group R is an alkyl group.

Even more preferably, the group R is chosen from the following groups: methyl, ethyl, butyl, isobutyl, propyl, isopropyl, octyl or allyl, which is substituted or unsubstituted. Preferably, the group R is a methyl group or an ethyl group.

The group R may optionally be substituted with one or more atoms or groups that do not interfere with the polymerization. Examples of such groups may be halogens (fluorine, chlorine, bromine, iodine), a cyano group, an alkoxy group or an acyloxy group.

The groups R⁸ and R⁹ represent, independently of each other, a hydrogen atom or a group comprising from 1 to 10 carbon atoms.

The groups R⁸ and R⁹ may be chosen independently of each other from the following groups: alkyl, cycloalkyl, aryl, arylalkyl, alkylaryl and a heterocyclic group comprising a ring comprising a heteroatom chosen from oxygen, sulfur and nitrogen. Alternatively, the groups R⁸ and R⁹ may represent a divalent group comprising from 3 to 10 carbon atoms. For example, the divalent group may comprise an aromatic ring comprising 5 or 6 carbon atoms.

Preferably, at least one from among R⁸ and R⁹, and preferably both R⁸ and R⁹ are a hydrogen atom.

As mentioned above, the polyvinyl ether compound may be a homopolymer. This homopolymer may be chosen from poly(methyl vinyl ether), poly(ethyl vinyl ether), poly(butyl vinyl ether), poly(isobutyl vinyl ether), poly(isopropyl vinyl ether), poly(propyl vinyl ether), poly(octyl vinyl ether).

Alternatively, the polyvinyl ether compound may be a copolymer comprising at least two different monomers of general formula [Chem 21]. In this case, the polyvinyl ether compound may be a copolymer comprising two different monomers of general formula [Chem 21] or three different monomers of general formula [Chem 21]. It is preferable for at least one of these monomers to be chosen from methyl vinyl ether or ethyl vinyl ether, and preferably for at least one of these monomers to be methyl vinyl ether.

The polyvinyl ether compound according to the invention may have a K value of from 30 to 120, and preferably from 40 to 70. The term “K value” means a measure of the mean degree of polymerization. The K value can be measured according to the standard ISO 1628-1.

The polyvinyl ether compound according to the invention may also have a glass transition temperature of from −60 to 0° C., and preferably from −50 to −5° C. The glass transition temperature was measured by differential scanning calorimetry (DSC). The glass transition temperature can be measured according to the standard NF EN ISO 11357-2.

Commercial polyvinyl ether compounds may include Lutonal® M 40, Lutonal® A 25, Lutonal® A 50, Lutonal® A 100, Lutonal® I 30, Lutonal® I 60, Lutonal® I 60 D and Lutonal® I 65 D available from the company BASF and Gantrez® M available from the company GAF.

The adhesive composition according to the invention may comprise from 1% to 60%, preferably from 5% to 40% and more preferably from 5% to 25% by weight of polyvinyl ether compound, relative to the total weight of the adhesive composition.

Curing Catalyst

The catalyst of the adhesive composition may be chosen from the group consisting of amines, organometallic compounds, acids and derivatives thereof, and mixtures thereof. It may be a mixture of catalysts of the same family (for example a mixture of several amines), or a mixture of catalysts of different families (for example a mixture of an amine and of an organometallic compound).

In the context of the invention, the term “organometallic compounds” means compounds comprising an organic radical and at least one metal.

In the context of the invention, the term “organic radical” means a radical comprising at least one carbon atom.

The organometallic compounds may comprise organometallic compounds (compounds comprising at least one metal-carbon covalent bond), metal alkoxides, metal carboxylates and metal coordination complexes with one or more organic ligands.

Examples of organic ligands that may be mentioned include acetylacetonate and oximes.

The metal atom of the organometallic compounds may be any metal atom known to those skilled in the art, and may be chosen in particular from tin, aluminum, zinc, cobalt, iron, nickel, bismuth, titanium, or zirconium. The organometallic compounds may moreover comprise several metal atoms.

The organometallic compounds (compounds comprising at least one metal-carbon covalent bond) may be carboxylates of organometallic compounds.

The organometallic compounds may be chosen from the group consisting of dibutyltin dilaurate (DBTL), dibutyltin diacetate, dibutyltin diethylhexanoate, dioctyltin dineodecanoate (available, for example, under the name TIB KAT® 223 from the company TIB Chemicals), dibutyltin dioleate, dibutyltin benzylmaleate, diphenyltin diacetate, and mixtures thereof.

The metal alkoxides may be chosen from the group consisting of titanium tetrabutoxide, titanium tetraisopropoxide, zirconium tetrabutoxide, zirconium tetraisopropoxide, and mixtures thereof.

The metal carboxylates may be chosen from the group consisting of zinc 2-ethylcaproate, zinc diacetate, zinc dineodecanoate, zinc diundecenoate, zinc di methacrylate, cobalt acetylacetonate, cobalt diacetate, iron acetylacetonate, iron diacetate, nickel acetylacetonate, nickel diacetate, bismuth acetate, bismuth trioctanoate, bismuth dineodecanoate, zinc bismuth dineodecanoate, and mixtures thereof.

The metal coordination complexes with one or more organic ligands may be chosen from the group consisting of zinc acetylacetonate, titanium acetylacetonate (commercially available, for example, under the name Tyzor® AA75 from the company Dorf Ketal), titanium tetraacetylacetonate, aluminum trisacetylacetonate, aluminum chelates, for instance bis(ethyl acetoacetate) monoacetylacetonate (commercially available, for example, under the name K-KAT® 5218 from the company King Industries), zirconium tetraacetylacetonate, diisopropoxybis(ethylacetonato)titanium, and mixtures thereof.

The amines may be primary amines, secondary amines or tertiary amines.

The amines may be aminosilanes, for instance aminopropyltrimethoxysilane, N-(2-aminoethyl)-3-aminopropyltrimethoxysilane, aminopropyltriethoxysilane, N-(2-aminoethyl)-3-aminopropyltriethoxysilane, bis(gamma-trimethoxysilylpropyl)amine, N-ethyl-gamma-aminoisobutyltrimethoxysilane, or N-phenyl-gamma-aminopropyltrimethoxysilane.

Preferably, the catalyst is not an aminosilane.

Preferably, the amines are chosen from the group consisting of triethylamine, tributylamine, tetramethylguanidine, 1,8-diazabicyclo[5.4.0]-7-undecene, 1,4-diazabicyclo[2.2.2]octane, 1,5-diazabicyclo[4.3.0]non-5-ene, N,N-bis(N,N-dimethyl-2-aminoethyl)methylamine, N,N-dimethylcyclohexylamine, N,N-dimethylphenylamine, N-ethylmorpholine, and mixtures thereof.

The acid catalysts may be chosen from organic acid catalysts, inorganic acid catalysts, and mixtures thereof.

Among the inorganic acid catalysts, examples that may be mentioned include phosphoric or orthophosphoric acid, phosphorous acid, hypophosphorous acid, or sulfuric acid.

Preferably, the organic acid catalysts have a pKa of less than or equal to 6, preferably less than or equal to 4, advantageously less than or equal to 2, advantageously less than or equal to 0.

The organic acid catalysts may be chosen from sulfonic acids, carboxylic acids, organophosphate acids, organophosphonate acids, phosphonic acids, and mixtures thereof.

The sulfonic acids may be aliphatic or aromatic, optionally substituted (for example substituted with at least one substituent chosen from halogens (such as fluorine), hydroxyls, alkyls, amines, and mixtures thereof), and may be mono- or disulfonic.

The sulfonic acids may be chosen from N-alkylaminoalkylsulfonic acids and N,N-dialkylaminoalkylsulfonic acids (zwitterions), for instance 2-(N-morpholino)ethanesulfonic acid, 3-(N-morpholino)propanesulfonic acid, 4-[N-morpholino]butanesulfonic acid, 1,4-piperazinediethanesulfonic acid, N-2-hydroxyethylpiperazine-N′-2-ethanesulfonic acid, 2-(N-morpholino)ethanesulfonic acid, N-morpholinomethanesulfonic acid, N-(2-hydroxyethyl)piperazine-N′-methanesulfonic acid, piperazine-N,N′-bis(methanesulfonic acid), cyclohexylaminomethanesulfonic acid, N-[tris(hydroxymethyl)methyl]aminomethanesulfonic acid, N,N-bis(2-hydroxyethyl)aminomethanesulfonic acid; para-toluenesulfonic acid; benzenesulfonic acid; methanesulfonic acid; dodecylbenzenesulfonic acid; dodecylbenzenedisulfonic acid; dinonylnaphthalenedisulfonic acid; dinonylnaphthalenesulfonic acid; trifluoromethylsulfonic acid; and mixtures thereof.

In particular, the sulfonic acids are chosen from para-toluenesulfonic acid, benzenesulfonic acid, methanesulfonic acid, dodecylbenzenesulfonic acid, dodecylbenzenedisulfonic acid, dinonylnaphthalenedisulfonic acid, dinonylnaphthalenesulfonic acid, trifluoromethylsulfonic acid, and mixtures thereof.

Among the carboxylic acid catalysts, examples that may be mentioned include malonic acid, succinic acid, maleic acid, oxalic acid, acetic acid, lactic acid, benzoic acid, citric acid, glycolic acid, and mixtures thereof.

In the context of the invention, and unless otherwise mentioned, the term “organophosphate acid” means a phosphoric acid ester comprising at least one —OH radical. For example, methyl phosphate is an organophosphate acid comprising two —OH radicals and having the general formula

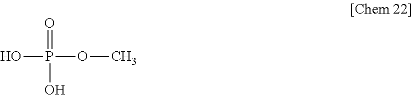

In particular, the organophosphate acids have the general formula [Chem 23]

(R¹⁰O)_{g}—(P═O)—(OH)ₕ
in which:
R¹⁰ is an organic radical, in particular a radical chosen from linear or branched C1-C22 alkyls, cycloalkyls, aryls, and mixtures thereof (said alkyl, cycloalkyl and aryl groups being optionally substituted); and
g and h are integers, with g+h=3 and h=1 or 2.

The organophosphate acids may be chosen, for example, from the group consisting of C1-C22 mono- or dialkyl phosphate acids and mixtures thereof, for instance butyl phosphate, dibutyl phosphate, bis(2-ethylhexyl) phosphate, 2-ethylhexyl phosphate, and mixtures thereof; mono- or diaryl phosphates, and mixtures thereof, for instance monophenyl phosphate, diphenyl phosphate and mixtures thereof; alkyl phenyl phosphates; and mixtures thereof.

In the context of the invention, and unless otherwise mentioned, the term “organophosphonate acid” means a phosphorus-based compound having the general formula [Chem 24]

R¹¹—(P═O)—(OH)(OR¹²)
in which R¹¹ and R¹² are organic radicals, preferably chosen, independently of each other, from linear or branched C1-C22 alkyls, cycloalkyls, aryls, and mixtures thereof (said alkyl, cycloalkyl and aryl groups being optionally substituted).

Among the organophosphonate acids, examples that may be mentioned include C1-C22 monoalkyl phosphonate acids.

In the context of the invention, and unless otherwise mentioned, the term “phosphonic acid” means a phosphorus-based compound having the general formula [Chem 25]

R¹³—(P═O)—(OH)₂

in which R¹³ is an organic radical, preferably chosen from linear or branched C1-C22 alkyls, cycloalkyls, aryls, and mixtures thereof (said alkyl, cycloalkyl and aryl groups being optionally substituted).

Among the phosphonic acids, examples that may be mentioned include N-alkylaminoalkylphosphonic acids (zwitterions), N,N-dialkylaminoalkylphosphonic acids (zwitterions), C1-C20 alkylphosphonic acids, for instance methylphosphonic acid, ethylphosphonic acid, propylphosphonic acid, butylphosphonic acid, t-butylphosphonic acid, isobutylphosphonic acid, hexylphosphonic acid, 2-ethylhexylphosphonic acid and linear or branched higher homologues, benzylphosphonic acid, phenylphosphonic acid, tolylphosphonic acid or xylylphosphonic acid.

Examples of organic acid catalysts that may be mentioned include Nacure® 155 (dinonylnaphthalenedisulfonic acid, containing 55% active material in isobutanol) sold by King Industries, Nacure® 1051 (dinonylnaphthalenesulfonic acid, containing 50% active material in 2-butoxyethanol) sold by King Industries, Nacure® 5076 (dodecylbenzenesulfonic acid, containing 70% active material in isopropanol) sold by King Industries, K-Cure® 1040 (para-toluenesulfonic acid, containing 40% active material in isopropanol) sold by King Industries, Nacure® 4000 (mixture of mono- and dialkyl phosphate acids, 100% active material) sold by King Industries.

The acid derivatives according to the invention may be acid anhydrides, acid esters or acid ammonium salts, the acid being as described above.

The acid derivatives are in particular “masked” or “latent” acids which advantageously make it possible to release the acid by thermal activation (for example at a temperature ranging from 70° C. to 170° C., preferably at a temperature ranging from 90° C. to 120° C.) or by hydrolysis, or by photoactivation, preferably by thermal activation.

The masked acid advantageously makes it possible to release the acid which is the species having the catalytic activity. For example, the ammonium salt formed between aminomethylpropanol and para-toluenesulfonic acid is a masked acid (acid derivative) which, by thermal activation, releases the para-toluenesulfonic acid.

The acid derivatives may be prepared via any means known to those skilled in the art starting with the corresponding acid, for example by using typical acid/base reactions. For example, the process for making an ester typically involves the condensation of an acid compound with a compound comprising a hydroxyl group, for instance an alcohol, or with a compound of oxirane type. The ammonium salts may be prepared from any abovementioned acid, with ammonia or with a primary, secondary or tertiary amine. The amines may optionally comprise at least one functional group such as a hydroxyl group (alkanolamines), a C1-C4 alkyl group. The ammonium salts (zwitterions) may also be prepared by modifying the pH of a solution containing, for example, N-alkylaminoalkylphosphonic acids, N,N-dialkylaminoalkylphosphonic acids, N-alkylaminoalkylsulfonic acids or N,N-dialkylaminoalkylsulfonic acids.

Preferably, the catalyst is an ammonium salt of a sulfonic acid (the sulfonic acid being as described above), an ammonium salt of a phosphonic acid (the phosphonic acid being as described above), an ammonium salt of an organophosphonate acid (the organophosphonate acid being as described above), or an ammonium salt of an organophosphate acid (the organophosphate acid being as described above).

As amines for the preparation of the ammonium salts, examples that may be mentioned include 2-amino-2-methyl-1-propanol, triethylamine, aniline, pyridine, dimethylaminoethanol, alkypyridines, diisopropanolamine, dimethylethanolamine, triethanolamine, oxazolidines, bicyclic oxazolidines, amidines, diazabicyclooctanes, guanidines, N-alkylmorpholines, aminopyridines, aminoalkylpyridines, aminopyrrolidines, indazole, imidazole, pyrazole, pyrazine, pyrimidine, purine, imidazoline, pyrazoline, piperazine, aminomorpholine, aminoalkylmorpholines, and mixtures thereof. Preferably, the amines are tertiary amines.

Examples of acid derivatives that may be mentioned include Nacure® 3327 or Nacure® 3525 (dinonylnaphthalenedisulfonic acid masked with an amine, containing 25% active material in isopropanol and isobutanol) sold by King Industries, Nacure® 1557 or Nacure® 1953 (dinonylnaphthalenesulfonic acid masked with an amine, containing 25% active material in a mixture of butanol and 2-butoxyethanol) sold by King Industries, Nacure® 5225 or Nacure® 5528 or Nacure® 5925 (dodecylbenzenesulfonic acid masked with an amine, containing 25% active material in isopropanol) sold by King Industries, Nacure® 2107 or Nacure® 2500 (para-toluenesulfonic acid masked with an amine, containing 25% or 26% active material in isopropanol) sold by King Industries, Nacure® 2501 or Nacure® 2530 (para-toluenesulfonic acid masked with an amine, containing 25% active material in a mixture of isopropanol and methanol) sold by King Industries, Nacure® 4167 (dialkyl phosphate masked with an organic amine, containing 25% active material in a mixture of isopropanol and isobutanol) sold by King Industries, Nacure® 4575 (phosphate acid blocked with an amine, containing 25% active material in a mixture of methanol and butanol) sold by King Industries.

Preferably, the catalyst is chosen from the group consisting of organometallic compounds (in particular aluminum-based coordination complexes), orthophosphoric acid, organophosphate acids (preferably C1-C22 mono- or dialkyl phosphate acid and mixtures thereof), ammonium salts (in particular of sulfonic acid or of organophosphate acid), and mixtures thereof. Even more preferably, the catalyst is chosen from the group consisting of orthophosphoric acid, organophosphate acids (preferably C1-C22 mono- or dialkyl phosphate acid and mixtures thereof), ammonium salts (in particular of sulfonic acid or of organophosphate acid).

The adhesive composition according to the invention may comprise from 0.1% to 4%, preferably from 0.2% to 2% and more preferably from 0.3% to 2% by weight of curing catalyst, relative to the total weight of the adhesive composition.

In the case where the composition according to the invention is a one-component composition, it is preferable to use one of the catalysts listed above other than inorganic acids and salts thereof, and also other than carboxylic acids.

In the case where the composition according to the invention is a two-component composition (as detailed below), the curing catalyst may be mixed in a reactive or non-reactive diluent. The term “non-reactive diluent” means a diluent which does not react in the presence of the catalyst. Thus, the term “reactive diluent” means a diluent that reacts in the presence of the catalyst. The diluent may be chosen from sparingly reactive monosilanes (Geniosil XM25, MS RD359), water scavengers (XL33) and silsesquioxane resins,

Non-reactive: polyols, tackifying resins, rheological modifiers (mentioned in other additives), etc.

This diluent may have a number-average molecular mass (Mn) ranging from 300 to 50 000 g/mol and preferably 1000 to 20 000 g/mol. This prevents migration of this diluent into the cured adhesive.

This diluent may also have a viscosity ranging from 10 to 100 000 mPa·s at 23° C. and even more preferentially from 500 to 15 000 mPa·s at 23° C.

This viscosity is measured with a Brookfield viscometer (cone and plate, CAP2000+).

Other Additives

The composition according to the present invention may comprise at least one other additive, chosen, for example, from the group consisting of tackifying resins, silsesquioxanes, plasticizers, solvents, pigments, dyes, adhesion promoters, moisture absorbers, UV stabilizers, antioxidants, glitter flakes, fluorescent materials, rheological additives, fillers, flame retardants, waxes, and mixtures thereof.

Tackifying Resin

The adhesive composition according to the invention may also comprise at least one tackifying resin.

The resin(s) used in the context of the invention may be any resin that is compatible with the silyl polymer(s).

The term “compatible tackifying resin” means a tackifying resin which, when mixed in 50/50 proportions by weight with the silyl polymer(s), gives a substantially homogeneous mixture (no visually observed phase separation).

In the context of the invention, the tackifying resin is different from the polyvinyl ether compound.

The tackifying resins are advantageously chosen from:
terpene-phenolic resins;
hydrocarbon resins;
rosin resins; and
acrylic resins.

The terpene-phenolic resins may have a softening point of from 85 to 150° C.

The hydrocarbon resins may have a softening point of from 15 to 140° C. The rosin resins may have a softening point of from 15 to 115° C. The softening point may be measured according to the standard ASTM E28.

The terpene-phenolic resins may be obtained by polymerization of terpene hydrocarbons and phenols in the presence of Friedel-Crafts catalyst(s).

The hydrocarbon resins may be chosen from: resins obtained via a process comprising the polymerization of α-methylstyrene, and optionally in the presence of phenols; resins obtained by hydrogenation, preferably partial hydrogenation, polymerization or copolymerization (with an aromatic hydrocarbon) of a mixture of unsaturated aliphatic hydrocarbons containing about 5, 9 or 10 carbon atoms derived from petroleum fractions, optionally grafted with maleic anhydride; terpene resins; generally resulting from the polymerization of terpene hydrocarbons, for instance monoterpene (or pinene) in the presence of Friedel-Crafts catalyst(s); copolymers based on natural terpenes, for instance styrene/terpene, α-methylstyrene/terpene and vinyltoluene/terpene; and mixtures thereof.

The rosin resins may be chosen from rosins of natural origin or modified rosins (for instance the rosin extracted from pine gum, wood rosin extracted from tree roots) and derivatives thereof which are hydrogenated, dimerized, polymerized or esterified with monoalcohols or polyols (for instance glycerol or pentaerythritol).

An acrylic resin is defined as a polymer or oligomer constructed with a significant amount of (meth)acrylic and/or (meth)acrylate monomers, preferably at least 5% weight/weight (w/w), more preferably at least 10% w/w, even more preferably at least 20% w/w, even more preferably at least 30% w/w in the polymer chain.

The (meth)acrylic monomers may be chosen from: acrylic acid, methacrylic acid, methyl acrylate, methyl methacrylate, ethyl acrylate, ethyl methacrylate, butyl acrylate, butyl methacrylate, isobutyl acrylate, isobutyl methacrylate, n-hexyl acrylate, n-hexyl methacrylate, 2-ethylhexyl acrylate, 2-ethylhexyl methacrylate, n-heptyl acrylate, n-heptyl methacrylate, stearyl acrylate, stearyl methacrylate, glycidyl methacrylate, alkyl crotonates, vinyl acetate, di-n-butyl maleate, dioctyl maleate, acetoacetoxyethyl acrylate, acetoacetoxyethyl methacrylate, acetoacetoxypropyl methacrylate, acetoacetoxypropyl acrylate, diacetoneacrylamide, acrylamide, methacrylamide, hydroxyethyl methacrylate, hydroxyethyl acrylate, allyl methacrylate, tetrahydrofurfuryl methacrylate, tetrahydrofurfuryl acrylate, cyclohexyl acrylate, cyclohexyl methacrylate, n-hexyl acrylate, n-hexyl methacrylate, 2-ethoxyethyl acrylate, 2-ethoxyethyl methacrylate, isodecyl methacrylate, isodecyl acrylate, 2-methoxy acrylate, 2-methoxy methacrylate, 2-(2-ethoxyethoxy)ethyl acrylate, 2-phenoxyethyl acrylate, 2-phenoxyethyl methacrylate, isobornyl acrylate, isobornyl methacrylate, caprolactone acrylate, caprolactone methacrylate, polypropylene glycol monomethacrylate, polypropylene glycol monoacrylate, polyethylene glycol acrylate (400), polypropylene glycol methacrylate (400), benzyl acrylate, benzyl methacrylate, N-vinylpyrrolidone or N-vinyllactam.

Preferably, the (meth)acrylic monomers contain up to 20 carbon atoms; more preferentially, the (meth)acrylic monomers are chosen from acrylic acid, methacrylic acid, butyl acrylate, 2-ethylhexyl acrylate and hydroxyethyl acrylate.

The acrylic resins may be chosen from polymers containing at least one (meth)acrylic function or chain part and at least one hydrocarbon-based chain part, said polymers being able to be in the form of copolymers, grafted or reacted or block copolymers.

The acrylic resins preferably have a viscosity at 100° C. of less than 100 Pa·s and less than or equal to 100 Pa·s at 150° C.

The acrylic resins may comprise repeating units of at least one hydrocarbon-based monomer and of at least one acrylate monomer.

The hydrocarbon-based monomers are chosen from the group consisting of styrene, α-methylstyrene, vinyltoluene, indene, methylindene, divinylbenzene, dicyclopentadiene and methyldicyclopentadiene, and polymerizable monomers contained in the C5-piperylene and C5-isoprene and C9-aromatic streams of the petrochemical industry. These hydrocarbon-based monomers are usually polymerized together in various ratios by cationic polymerization using Lewis acid catalysts.

The acrylate monomers are chosen from methyl acrylate, acrylic acid, methacrylic acid, methyl methacrylate, ethyl acrylate, ethyl methacrylate, butyl acrylate, butyl methacrylate, isobutyl acrylate, isobutyl methacrylate, n-hexyl acrylate, n-hexyl methacrylate, ethylhexyl acrylate, ethylhexyl methacrylate, n-heptyl acrylate, heptyl methacrylate, 2-methylheptyl (meth)acrylate, octyl acrylate, octyl methacrylate, isooctyl (meth)acrylate, n-nonyl (meth)acrylate, isononyl (meth)acrylate, decyl (meth)acrylate, isodecyl acrylate, isodecyl (meth)acrylate, dodecyl (meth)acrylate, isobornyl (meth)acrylate, lauryl acrylate, lauryl (meth)acrylate, tridecyl acrylate, tridecyl methacrylate, stearyl acrylate, stearyl methacrylate, glycidyl methacrylate, alkyl crotonates, vinyl acetate, di-n-butyl maleate, dioctyl maleate, acetoacetoxyethyl methacrylate, acetoacetoxyethyl acrylate, acetoacetoxypropyl methacrylate, acetoacetoxypropyl acrylate, diacetoneacrylamide, acrylamide, methacrylamide, hydroxyethyl methacrylate, hydroxyethyl acrylate, allyl methacrylate, tetrahydrofurfuryl methacrylate, tetrahydrofurfuryl acrylate, cyclohexyl methacrylate, cyclohexyl acrylate, n-hexyl acrylate, n-hexyl methacrylate, 2-ethoxyethyl acrylate, 2-ethoxyethyl methacrylate, isodecyl methacrylate, isodecyl acrylate, 2-methoxy acrylate, 2-methoxy methacrylate, 2-(2-ethoxyethoxy)ethyl acrylate, 2-phenoxyethyl acrylate, 2-phenoxyethyl methacrylate, isobornyl acrylate, isobornyl methacrylate, caprolactone acrylate, caprolactone methacrylate, polypropylene glycol monoacrylate, polypropylene glycol monomethacrylate, polyethylene glycol (400) acrylate, polypropylene glycol (400) methacrylate, benzyl acrylate, benzyl methacrylate, sodium 1-allyloxy-2-hydroxypropyl sulfonate, acrylonitrile and mixtures thereof.

Preferably, the hydrocarbon-based monomers are chosen from the group of aromatic monomers or polymerizable monomers from the C9 aromatic stream from petrochemical sources; dicyclopentadiene or polymerizable monomers from the C5-piperylene or C5-isoprene stream from petrochemical sources.

Preferably, the acrylate monomers are chosen from acrylic acid, 2-ethylhexyl acrylate, hydroxyethyl acrylate, methacrylic acid, butyl acrylate.

The softening point of these acrylic resins is preferably from room temperature to 180° C., preferably less than or equal to 150° C., more preferably less than or equal to 120° C., and even more preferably from 70 to 120° C. The softening point may be measured according to the standard ASTM E28.

Such resins are commercially available, and examples that may be mentioned include the following products:

For the resins obtained by polymerization of terpene hydrocarbons and phenols, in the presence of Friedel-Crafts catalyst(s): Dertophene® 1510 available from the company DRT, with a number-average molecular mass Mₙ of about 870 Da; Dertophene® H150 available from the company DRT, with a number-average molecular mass Mₙ of about 630 Da; Dertophene® T available from the company DRT, with a number-average molecular mass Mₙ of about 500 Da; Sylvarez® TP 95 available from the company Arizona Chemical, with a number-average molecular mass of about 1200 Da.

For the resins obtained via a process comprising the polymerization of α-methylstyrene, optionally in the presence of phenols: Cleartack® W100 available from the company Cray Valley, which is obtained by polymerization of α-methylstyrene without action of phenols, with a number-average molecular mass of 900 Da; Sylvarez® 510, which is available from the company Arizona Chemical, with a number-average molecular mass of about 1740 Da, the process for the production of which comprises the addition of phenols.

For the rosins of natural origin or modified rosins: Sylvalite® RE 100, which is an ester of rosin and of pentaerythritol available from the company Arizona Chemical, and the number-average molecular mass of which is about 1700 Da.

According to a preferred embodiment, the tackifying resin is chosen from resins obtained by polymerization of terpene hydrocarbons and phenols, in the presence of Friedel-Crafts catalyst(s).

The tackifying resin preferably has a number-average molecular mass ranging from 100 to 6000 g/mol, preferably from 300 to 4000 g/mol, preferentially from 500 to 2000 g/mol.

The number-average molecular masses of the tackifying resins may be measured by methods that are well known to those skilled in the art, for example by size exclusion chromatography using a polystyrene-type standard.

The tackifying resin may have a hydroxyl number I_{OH} ranging from 10 to 300 mg KOH/g, preferably ranging from 100 to 200 mg KOH/g, preferentially ranging from 140 to 160 mg KOH/g. In particular, the tackifying resin has a hydroxyl number of 145 mg KOH/g.

The hydroxyl number of the tackifying resin represents the number of hydroxyl functions per gram of tackifying resin, and is expressed in the form of the equivalent number of milligrams of potassium hydroxide per gram of tackifying resin (mg KOH/g) for the assay of the hydroxyl functions.

The acid number of the rosin ester tackifying resin may range from 0 to 10 mg KOH/g, and preferentially from 0 to 5 mg KOH/g. The acid number of the tackifying resin represents the number of acid functions per gram of tackifying resin, and is expressed in the form of the equivalent number of milligrams of potassium hydroxide per gram of tackifying resin (mg KOH/g) for the assay of the acid functions.

The adhesive composition according to the invention may comprise from 0.1% to 80%, preferably from 20% to 70% and more preferably from 30% to 60% by weight of tackifying resin, relative to the total weight of the adhesive composition.

In the case where the composition according to the invention comprises at least one tackifying resin, the mass ratio of polyvinyl ether compound to tackifying resin may be from 3% to 100%.

According to certain embodiments, the adhesive composition comprises a single tackifying resin.

According to alternative embodiments, the adhesive composition comprises different tackifying resins, for example two or three or four or five different tackifying resins.

Silsesquioxanes

Silsesquioxanes are typically organosilicon compounds which can adopt a polyhedral structure or a polymeric structure, with Si—O—Si bonds. They typically have the general formula [Chem 26]

[R′SiO_{3/2}]ₜ
in which R′, which may be identical or different in nature, represents an organic radical and t is an integer which may range from 6 to 12, t preferably being equal to 6, 8, 10 or 12.

According to one embodiment, the silsesquioxane has a polyhedral structure (or POSS for “Polyhedral Oligomeric Silsesquioxane”).

Preferably, the silsesquioxane corresponds to the general formula

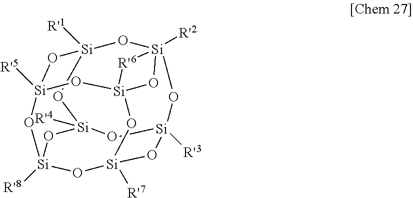

in which each one from among R′¹ to R′⁸ represents, independently of each other, a group chosen from:
a hydrogen atom;
a radical chosen from the group consisting of a linear or branched C1-C4 alkoxy radical, a linear or branched alkyl radical comprising from 1 to 30 carbon atoms, an alkenyl radical comprising from 2 to 30 carbon atoms, an aromatic radical comprising from 6 to 30 carbon atoms, an allyl radical comprising from 3 to 30 carbon atoms, a cyclic aliphatic radical comprising from 3 to 30 carbon atoms and an acyl radical comprising from 1 to 30 carbon atoms; and
a group —OSiR′⁹R′¹⁰ in which R′⁹ and R′¹⁰ each represent, independently of each other, a hydrogen atom or a radical chosen from the group consisting of linear or branched C1-C4 alkyls, linear or branched C1-C4 alkoxys, C2-C4 alkenyls, a phenyl, a C3-C6 allyl radical, a cyclic C3-C8 aliphatic radical and a C1-C4 acyl radical;
on condition:
that at least one radical from among the radicals R′¹ to R′⁸ is a C1-C4 alkoxy radical; and that at least one radical from among the radicals R′¹ to R′⁸ is a phenyl radical.

Silsesquioxanes are known compounds that are notably described in patent application WO 2008/107331 A1. Some are also commercially available, such as the product from Dow sold under the name: Dow Corning® 3074 and Dow Corning® 3037 (CAS number=68957-04-0).

The adhesive composition according to the invention may comprise from 0 to 40%, preferably from 0 to 20% and more preferably from 0 to 10% by weight of silsesquioxane, relative to the total weight of the adhesive composition.

Fillers

The filler may be chosen from organic fillers, inorganic fillers and mixtures thereof.

As organic filler(s), use may be made of any organic filler and notably polymeric fillers typically used in the field of adhesives. Use may be made, for example, of polyvinyl chloride (PVC), polyolefin(s), rubber, ethylene vinyl acetate (EVA), aramid fibers such as Kevlar®, expandable or non-expandable thermoplastic polymer hollow microspheres (for instance hollow microspheres made of vinylidene chloride/acrylonitrile), thermoplastic polymer(s) chosen from those used in the preparation of HMPSA, such as ethylene vinyl acetate (EVA), or styrene block copolymers (such as SIS, SBS, SIBS, SEBS, SEPS, and derivatives thereof grafted, for example, with maleic anhydride).

The filler may be an expander (also known as a swelling agent).

The filler may be in the form of hollow beads, i.e. beads containing a gas, or of beads that can be expanded to form hollow beads, i.e. beads containing a void or a gas.

Preferably, the filler is an inorganic filler.

According to one embodiment, the filler is chosen from sand, precipitated and/or fumed silica, zeolites, glass beads, glass, quartz, barite, alumina, mica, talc, alkali metal or alkaline-earth metal carbonates (for example calcium carbonate).

The filler(s) preferably represent from 0% to 15% by weight, preferably from 0% to 10% by weight, preferentially from 0% to 5% by weight, relative to the total weight of the adhesive composition.

According to one embodiment, the composition according to the invention does not comprise any filler.

Plasticizer

The composition according to the invention may comprise at least one plasticizer. The total content of plasticizer(s) in the composition may range from 0% to 30% by weight, preferably from 1% to 30% by weight or even, for example, from 1% to 15% by weight relative to the total weight of said composition.

As examples of plasticizers that may be used, use may be made of any plasticizer usually used in the field of adhesives, for instance phthalates, benzoates, trimethylolpropane esters, trimethylolethane esters, trimethylolmethane esters, glycerol esters, pentaerythritol esters, naphthenic mineral oils, adipates, cyclohexyldicarboxylates, liquid paraffins, natural oils (optionally epoxidized), polypropylenes, polybutylenes, hydrogenated polyisoprenes, and mixtures thereof.

Among the phthalates, examples that may be mentioned include diisononyl phthalate, diisobutyl phthalate, dioctyl phthalate, dicyclohexyl phthalate, diisooctyl phthalate, diisododecyl phthalate, dibenzyl phthalate or butylbenzyl phthalate.

Among the benzoates, examples that may be mentioned include: neopentyl glycol dibenzoate (available, for example, under the name Uniplex® 512 from Lanxess), dipropylene glycol dibenzoate (available, for example, under the name Benzoflex® 9-88SG from Eastman), a mixture of diethylene glycol dibenzoate and of dipropylene glycol dibenzoate (available, for example, under the name K-Flex® 850 S from Kalama Chemical), or a mixture of diethylene glycol dibenzoate, dipropylene glycol dibenzoate and triethylene glycol dibenzoate (available, for example, under the name Benzoflex® 2088 from Eastman).

Among the pentaerythritol esters, examples that may be mentioned include pentaerythrityl tetravalerate (available, for example, under the name Pevalen™ from the company Perstorp).

Among the cyclohexanedicarboxylates, an example that may be mentioned is diisononyl 1,2-cyclohexanedicarboxylate (available, for example, under the name Hexamoll Dinch® from BASF).

Pigment

When a pigment is present in the composition according to the invention, its content is preferably less than or equal to 3% by weight, more preferably less than or equal to 2% by weight, relative to the total weight of the composition.

When it is present, the pigment may represent, for example, from 0.1% to 3% by weight or from 0.4% to 2% by weight relative to the total weight of the composition according to the invention.

The pigments may be organic or inorganic pigments.

For example, the pigment is TiO₂, in particular Kronos® 2059 sold by the company Kronos.

Moisture Absorber

The moisture absorber, if it is present, may be chosen, for example, from non-polymeric hydrolyzable alkoxysilane derivatives, with a molecular mass of less than 500 g/mol, preferably chosen from trimethoxysilane and triethoxysilane derivatives. Such an agent may typically extend the shelf life of the composition during storage and transportation before it is used.

Mention may be made, for example, of γ-methacryloxypropyltrimethoxysilane (for example available, under the trade name Silquest® A-174, from the company Momentive), methacryloxymethyltrimethoxysilane (for example available, under the name Geniosil® XL33, from Wacker), vinyltrimethoxysilane, isooctyltrimethoxysilane or phenyltrimethoxysilane.

The moisture absorber content is preferably less than or equal to 3% by weight, more preferably less than or equal to 2% by weight relative to the total weight of the composition according to the invention. When present, the moisture absorber may represent, for example, from 0.1% to 3% by weight or from 1% to 2% by weight relative to the total weight of the composition.

The composition according to the invention may comprise an amount of from 0.1% to 3%, preferably from 1% to 3%, by weight of at least one UV stabilizer or antioxidant. These compounds are typically introduced to protect the composition from degradation resulting from a reaction with oxygen which is liable to be formed by the action of heat or light. These compounds may include primary antioxidants which trap free radicals. The primary antioxidants may be used alone or in combination with other secondary antioxidants or UV stabilizers.

Examples that may be mentioned include Irganox® 1010, Irganox® B561, Irganox® 245 and Irganox® 168 sold by BASF.

One-Component or Two-Component Composition

According to one embodiment, the adhesive composition according to the invention is a one-component composition. In other words, it is a composition in which all the compounds are packaged in the same compartment. In this case, the composition is preferably ready to use, i.e. the user (private individual or professional) can apply the adhesive composition directly to the substrate to be covered and/or the flexible covering, without having to perform any prior mixing.

According to alternative embodiments, the adhesive composition according to the invention is a two-component composition. In other words, it is a composition in which the components are packaged in two separate compartments.

In this case, the two-component composition may comprise a part A and a part B, the two parts being mixed prior to use and application of the adhesive composition to a substrate layer, for example. The components described above may be distributed in part A and/or in part B of the two-component composition.

According to preferred embodiments, part A of the two-component composition may comprise, for example, the silyl polymer (as described above). Part B of the composition may in this case comprise the curing catalyst (as described above). The polyvinyl ether compound and also optional additives may be present in part A and/or part B of the composition.

The additional components (other additives) may be present in part A and/or part B of the two-component composition.

Use of the Composition

The composition according to the invention is used for manufacturing self-adhesive articles. The term “self-adhesive article” means any article that can be adhesively bonded to a surface solely by the action of pressure with the hand or an item of equipment, without the use of additional glues or adhesives.

Self-adhesive articles may comprise a support layer coated with a self-adhesive layer, said self-adhesive layer being the adhesive composition according to the invention in cured form.

Preferably, the self-adhesive article is a pressure-sensitive self-adhesive article.

These articles notably have the aim of being applied to a surface to be bonded so as to bring together, maintain, fix, or simply immobilize, expose forms, logos, images or information. These articles may be used in many fields, such as the medical field, clothing, packaging, motor vehicles (for example for attaching logos, lettering, interior soundproofing, interior trim, bonding in the passenger compartment) or construction (for example for sound and thermal insulation, the assembling of windows); in particular in the medical sector and in the construction sector. They may be fashioned as a function of their final application, for example in the form of tapes, such as tapes for industrial use, tapes for do-it-yourself work or for fixing use on worksites, single-sided or double-sided tapes, or in the form of labels, bandages, dressings, patches or graphic films.

According to one embodiment, the self-adhesive article is a self-adhesive multilayer system, and in particular a self-adhesive label or tape, which may be single-sided or double-sided.

The material that may be used for the support layer may be, for example, any type of rigid or flexible support. Examples that may be mentioned include supports of the type such as foams, felts, nonwoven support, plastics, membranes, papers or a film of a polymer material with one or more layers.

The support layer is made of a material chosen, for example, from polyolefins, such as polyethylene, including high-density polyethylene, low-density polyethylene, linear low-density polyethylene and linear ultra-low-density polyethylene; polypropylene and polybutylenes; polystyrene; natural or synthetic rubber; vinyl copolymers, such as polyvinyl chloride, which may or may not be plasticized, and poly(vinyl acetates); olefinic copolymers, such as ethylene/methacrylate copolymers, ethylene/vinyl acetate copolymers, acrylonitrile/butadiene/styrene copolymers, and ethylene/propylene copolymers; acrylic polymers and copolymers; polyurethanes; polyethers; polyesters; and mixtures thereof. Preferably, the support layer is based on acrylic polymers, polyethylene (PE), polypropylene (PP), which may be oriented, non-oriented or bioriented, polyimide, polyurethane, polyester such as polyethylene terephthalate (PET), or paper.

According to certain embodiments, the self-adhesive article obtained from the adhesive composition according to the invention comprises a permanent support layer coated with an adhesive layer. Preferably, the adhesive layer is also coated with a nonstick protective paper or plastic film, which is preferably silicone-treated.

As an alternative to the nonstick protective film, the rear face of the permanent support layer, which is not coated with the adhesive layer, may have a nonstick surface, for example a silicone-treated protective layer.

According to one embodiment, the permanent support layer is coated on both its faces with an adhesive composition, which can be identical or different, at least one of the two adhesive compositions being according to the invention.

Preferably, the support layer has a thickness ranging from 10 μm to 50 mm, more preferably ranging from 10 μm to 20 mm, preferably ranging from 20 μm to 10 mm, more preferably ranging from 20 μm to 1 mm.

In certain specific cases, it is necessary to perform a surface treatment on the support layer to increase the attachment of the adhesive layer during the step of coating thereon.

The self-adhesive article according to the invention can thus bond two substrates. The substrate onto which the self-adhesive article is intended to be applied (referred to as the “substrate to be bonded”) may be flexible or rigid. In particular, it may have the same flexibility properties as the support layer described above, so as to be rolled up and packaged in the form of a reel. Alternatively, the substrate to be bonded may be rigid. In this case, the substrate cannot be rolled up and packaged in the form of a reel, for example as described previously. The substrate to be bonded may be chosen, for example, from concrete, paper, substrates of polyolefin type, etc.

According to certain embodiments, the self-adhesive article also comprises a protective nonstick layer (“release liner”).

According to certain embodiments, said nonstick layer is applied to the adhesive layer, after curing the adhesive composition.

The support layer may be covered on one of its two faces, the rear face which is not coated with the adhesive layer, with a protective nonstick layer, for example with a silicone film. In this way, the self-adhesive article can be wound up on itself and then unwound without any problem by virtue of the absence of adhesion of the adhesive layer to the silicone-treated face.

The self-adhesive article according to the invention is able to be obtained via the process comprising the following steps:
mixing parts A and B of the adhesive composition according to the invention, in the case where it is a two-component composition;
heating the adhesive composition to a temperature ranging from 40° C. to 130° C.;
coating a bearing surface with the adhesive composition;
curing the coated adhesive composition, by heating to a temperature ranging from 50° C. to 200° C. in particular in a gaseous environment in which water molecules are present at between 10 and 200 g per m³ of gas;
counter-bonding or transfer of the cured adhesive layer onto a support layer or onto a nonstick protective film, said support layer or nonstick film possibly being the reverse side of the bearing surface.

For the purposes of the present invention, the term “bearing surface” should be understood as meaning either a belt conveyor coated with a nonstick layer, or a nonstick protective film (“release liner”), or a support layer. Thus, the bearing surface is made to become an integral part of the self-adhesive article, either as nonstick protective film, or as support layer.

In the case where the bearing surface is not a support layer, the last step of the above process comprises the transfer of the cured adhesive layer onto a support layer.

In the case where the bearing surface is a support layer, the last step of the above process comprises the counter-bonding of the adhesive layer onto a nonstick protective film.

According to a preferred variant of the invention, the last step of the above process consists in transferring the cured adhesive layer onto a flexible support layer (which may be a plastic film) after cooling of the cured adhesive layer to a temperature below the degradation temperature or softening point of the material of which the support layer is composed.

According to one embodiment, the self-adhesive article according to the invention may be obtained via the process as described previously, not comprising a step of pretreatment of the surface of the support layer. These pretreatments are directed toward chemically and/or physically modifying said surface, to increase the surface energy and/or the roughness of said surface, and thus to improve the adhesion of the adhesive layer to said surface. By way of example of known surface treatments, mention may be made of a plasma, corona treatment, abrasion or application to said surface of a chemical attachment agent (also known as a primer) which is capable of giving the substrate coated with said agent a high surface energy.

The process for manufacturing the self-adhesive article according to the invention may also comprise a step of coating a second layer of adhesive composition according to the invention onto the support layer followed by a step of curing the coated adhesive composition by heating to a temperature ranging from 20 to 200° C. According to this embodiment, a double-sided self-adhesive article is obtained.

The coating step(s) may be performed by means of known coating devices, for instance a lip nozzle or a nozzle of curtain type, or else with a roller.

These steps may use a weight per unit area of adhesive composition ranging from 3 to 5000 g/². The weight per unit area of adhesive composition required for the manufacture of self-adhesive labels may range from 10 to 100 g/m², and preferably from 20 to 50 g/m². The weight per unit area required for the manufacture of self-adhesive tapes may vary within a much wider range extending from 3 to 5000 g/m², and preferably from 15 to 250 g/m² per face.

According to certain embodiments, the coated adhesive composition is also subjected, during the curing step, to a treatment in a humid atmosphere characterized by its humidity level. Preferably, the humid atmosphere is an atmosphere in which from 2% to 100% of the molecules are water molecules, preferably from 3% to 50%, more preferably from 3% to 10% of the molecules are water molecules.

The moisture content is expressed as the percentage of water per unit volume, which corresponds to the number of water molecules divided by the total number of molecules in a unit of volume. By virtue of the linear nature of this scale, the moisture content is readily measured and monitored by using, for example, monitors of P.I.D (Proportional-Integral-Derivative) type. The weight percentage may be calculated by multiplying the percentage of the number of water molecules relative to the total number of molecules by a factor of 0.622. General information regarding the moisture content in various environments is described by W. Wagner et al. in International Steam Tables—Properties of Water and Steam based on the Industrial Formulation IAPWS-IF97.

The thermal curing step has the effect notably of creating—between the polymer chains bearing hydrolyzable alkoxysilane end groups of the adhesive composition and under the action of atmospheric moisture—bonds of siloxane type which lead to the formation of a three-dimensional polymer network. The adhesive composition thus cured is in particular a pressure-sensitive adhesive which gives the support layer which is coated therewith the desirable adhesive power and tack.

Preferably, the coating is performed uniformly over the support layer or over the non-stick protective layer, but the coating may also be adapted to the desired shape of the final self-adhesive article.

According to one embodiment, coating with the adhesive composition is performed over at least a portion of the two faces of the support layer. If the two faces of the support layer are coated, the adhesive composition may be identical or different on the two faces and the weight per unit area may be identical or different on the two faces.

According to certain embodiments of the invention, the self-adhesive article comprises an adhesive layer on at least a portion of one face or on at least a portion of the two faces of the support layer, said adhesive layer(s) being optionally coated with a nonstick protective layer. According to one embodiment, the self-adhesive article comprises two nonstick protective layers on each of the two adhesive layers. In this case, the two protective layers can be made of identical or different materials and/or they may have an identical or different thickness.

The self-adhesive articles according to the present invention are waterproof breathable articles. The term “waterproof breathable” means permeable to water vapor and impermeable to liquid water.

Preferably, the article obtained from the composition according to the invention has a permeability to water vapor (MVTR, for “Moisture Vapor Transmission Rate”) of at least 350 g/m² per 24 hours, at 37° C., at a relative humidity of 50%, for a film thickness of 30 μm. More preferably, the permeability to water vapor MVTR of the film is at least 500 g/m²/24 h, preferably at least 600 g/m²/24 h, more preferably at least 700 g/m²/24 h, and more preferably 800 g/m²/24 h, at 37° C., at a relative humidity of 50%, for a film thickness of 30 μm. In particular, the MVTR membrane permeability may range from 350 to 400 g/m²/24 h, or from 400 to 500 g/m²/24 h, or from 500 to 600 g/m²/24 h, or from 600 to 700 g/m²/24 h, or from 700 to 800 g/m²/24 h, or from 800 to 900 g/m²/24 h, or from 900 to 1000 g/m²/24 h, or from 1000 to 1200 g/m²/24 h, or from 1200 to 1500 g/m²/24 h, or from 1500 to 2000 g/m²/24 h, or from 2000 to 2500 g/m²/24 h, or from 2500 to 3000 g/m²/24 h, or from 3000 to 3500 g/m²/24 h, or from 3500 to 4000 g/m²/24 h, or from 4000 to 4500 g/m²/24 h, or from 4500 to 5000 g/m²/24 h, at 37° C., at a relative humidity of 50%, for a film thickness of 30 μm. The permeability to water vapor (MVTR) of the film, at 37° C., for a relative humidity of 50%, for a film thickness of 30 μm, may be measured according to the standard ASTM E96 B. The self-adhesive article according to the invention can be used in a bonding method comprising the following steps:
removing the nonstick protective layer, when such a layer is present;
applying the self-adhesive article to one surface of a product; and
applying pressure to said article.

In the second step, the self-adhesive article is applied so that the self-adhesive part of the article (formed by the self-adhesive layer) is facing the surface of the product.

According to certain embodiments in which the self-adhesive article is a double-sided article, the bonding method also comprises a step in which either a second surface of a product is applied to the article bonded to the first surface of a product, or the article bonded to the first surface of a product is applied to a second surface of a product.

### EXAMPLES

The following examples illustrate the invention without limiting it.

“PSA” Adhesion Performance

The adhesive compositions were applied to a polyethylene terephthalate (PET) substrate layer to form a layer 50 μm thick.

The substrate layers coated with the adhesive compositions were stored for 7 days at 70° C. to ensure complete curing of the composition.

The adhesion performance of the compositions was measured by a 180° peel test according to the standard Finat 1. Thus, according to this standard, ⅔ of an adhesive specimen (15 cm×2.5 cm) is placed on a test plate (sanded stainless steel, HDPE etc.). A 2 kg roller is passed over the specimen twice to promote wettability. A waiting time of 10 minutes is observed before the traction. The specimen is positioned parallel to the traction, and is bent back on itself at an angle of 180° C. The part of the specimen not bonded to the test plate provides a grip for the jaw and thus allows traction at a standard speed of 300 mm/min. The device measures the force required to detach the specimen under the conditions described previously.

The principle of this test is to determine the force required to separate (or peel) the adhesive composition layer from the substrate layer.

The tack of the compositions was measured according to the standard Finat 9. Thus, according to this standard, a 15 cm×2.5 cm adhesive specimen is folded into a loop with the adhesive on the outer face of the loop. At a speed of 300 mm/min, the loop is brought together and then gently placed on a plate (glass). Once a 1 inch² square is placed on the plate, the loop is removed from the plate and the force required to remove the adhesive is measured.

Water Vapor Permeability (MVTR)

This test measures the amount of water vapor that can pass through the adhesive composition layer.

This test was performed by applying the standard NF EN 13726-2 at 37° C. The adhesive compositions were applied to a substrate layer of nonwoven (NW) material with an MVTR of 5861 g/m²/24 h or to a substrate layer of polyurethane (PU) with an MVTR of 7826 g/m²/24 h, so as to form a layer with a thickness of 0.03 mm without the backing (support for reinforcing the PU film during lamination, thickness of 0.104 mm with backing) The substrate layers coated with the adhesive compositions were stored for 7 days at 70° C. to ensure complete curing of the composition.

### Example 1

The following components were used to prepare a composition A (according to the invention) and a composition B (reference):
Cleartack® W100, available from the company Cray Valley, is a tackifying resin obtained by polymerization of α-methylstyrene without the action of phenols, having a number-average molecular mass of 900 Da;
Sylvalite® RE 100, available from the company Arizona Chemical, is a rosin-type resin with a number-average molecular mass of about 1700 Da;
Lutonal® M40, available from BASF, is a polyvinyl ether;
Irganox® 1010, available from BASF, is an antioxidant of hindered phenol type;
SPUR Y-19204, available from the company Momentive, is a silyl polypropylene glycol polyurethane prepolymer bearing trimethoxysilane end groups and having an average molecular weight Mn of 24 000 g/mol;
K-KAT® 5218, available from the company King Industries, is a catalyst of aluminum chelate type.

TABLE 1

| Compositions (%) | A (inv) | B (ref) |
|---|---|---|
| Cleartack ® W100 | 18.48 | 23.43 |
| Sylvalite ® RE 100 | 18.48 | 23.43 |
| Lutonal ® M40 | 9.90 | — |
| Irganox ® 1010 | 0.50 | 0.50 |
| Spur Y-19204 | 51.64 | 51.64 |
| K-KAT ® 5218 | 1.00 | 1.00 |

Composition B is free of polyvinyl ether compound.

The adhesion performance and the vapor permeability were measured as detailed above. The results are illustrated in the table below:

TABLE 2

| Performance at 50 gsm | | | | |
|---|---|---|---|---|
| Compositions | A (inv) | | B (ref) | |
| 180° peel SS (N/inch)/PET | 15.2 ± | 0.51 AF | 17.2 ± | 0.13 AF |
| 180° peel HDPE (N/inch)/PET | 10.3 ± | 0.74 AF | 15.4 ± | 0.35 AF |
| “Tack” power (N/inch)/PET | 19.2 ± | −1.4 AF | 18.5 ± | 2.4 AF |
| MVTR (g/m²/24 h)/NW | 1107 ± | 51 | 865 ± | 60 |

Key:
SS=polished stainless steel
AF=Adhesive Failure (no residue left, loss of adhesion explains the test result, product has good cohesion)
HDPE=high density polyethylene

It is seen that composition A according to the invention makes it possible to obtain articles with improved vapor permeability, without, however, compromising the good adhesion properties.

### Example 2

The following components were used to prepare a composition C (according to the invention) and a composition D (reference):
Picco® AR100, available from the company Eastman, is a hydrocarbon-based resin;
Lutonal® M40, available from BASF, is a polyvinyl ether;
Geniosil® STPE-30, available from the company Wacker, is a dimethoxy(methyl)silylmethylcarbamate-terminated polyether with an average molecular weight of 24 000 g/mol;
K-KAT® 5218, available from the company King Industries, is a catalyst of aluminum chelate type.

TABLE 3

| Compositions (%) | C (inv) | D (ref) |
|---|---|---|
| Picco ® AR100 | 44.14 | 53.36 |
| Lutonal ® M40 | 10.22 | — |
| Geniosil ® STPE-30 | 44.64 | 44.64 |
| K-KAT ® 5218 | 1.00 | 1.00 |

Composition D is free of polyvinyl ether compound.

The adhesion performance and the vapor permeability were measured as detailed above. The results are illustrated in the table [table 4] below:

TABLE 4

| Compositions | | | | |
|---|---|---|---|---|
| Performance at 50 gsm | C (inv) | | D (ref) | |
| 180° peel SS (N/inch)/PET | 32.5 ± | 0.85 AF | 34.2 ± | 2.5 AF |
| 180° peel HDPE high density | 22.2 ± | 0.70 AF | 24.7 ± | 0.34 AF |
| polyethylene, (N/inch)/PET | | | | |
| “Tack” power (N/inch)/PET | 39.7 ± | −2.9 AF | 48.4 ± | 1.0 AF |
| MVTR (g/m²/24 h)/NW | 772 ± | 32 | 488 ± | 118 |

It is seen that composition C according to the invention makes it possible to obtain articles with improved vapor permeability, without, however, compromising the good adhesion properties.

### Example 3

The following components were used to prepare a composition E (according to the invention) and a composition F (comparative):
Cleartack® W100, available from the company Cray Valley, is a tackifying resin obtained by polymerization of α-methylstyrene without the action of phenols, having a number-average molecular mass of 900 Da;
Sylvalite® RE 100, available from the company Arizona Chemical, is a rosin-type resin with a number-average molecular mass of about 1700 Da;
Lutonal® M40, available from BASF, is a polyvinyl ether;
Irganox® 1010, available from BASF, is an antioxidant of hindered phenol type;
Spur Y-19204, available from the company Momentive, is a silyl polypropylene glycol polyurethane prepolymer bearing trimethoxysilane end groups and having an average molecular weight Mn of 24 000 g/mol;
TnBT is a titanium tetrabutoxide catalyst.

TABLE 5

| Compositions (%) | E (inv) | F (comp) |
|---|---|---|
| Cleartack ® W100 | 13.5 | 23.4 |
| Sylvalite ® RE 100 | 13.5 | 23.4 |
| Lutonal ® M40 | 19.8 | — |
| Irganox ® 1010 | 0.5 | 0.5 |
| Spur Y-19204 | 51.6 | 51.6 |
| TnBT | 1 | 1 |

Composition F is free of polyvinyl ether compound.

The adhesion performance and the vapor permeability were measured as detailed above. The results are illustrated in the table below:

TABLE 6

| Compositions | | | | |
|---|---|---|---|---|
| 150 gsm | E (inv) | | F (comp) | |
| 180° peel SS (N/inch)/PET | 16.9 ± | 0.1 AF | 18.1 ± | 0.4 AF |
| MVTR (g/m²/24 h)/PU | 395.6 ± | 6.04 | 215.6 ± | 2.77 |

It is seen that composition E according to the invention makes it possible to obtain articles with improved vapor permeability, without, however, compromising the good adhesion properties.

1-15. (canceled)

16. An adhesive composition comprising:
at least one silyl polymer comprising at least one hydrolyzable alkoxysilane group;
at least one polyvinyl ether compound; and
at least one curing catalyst.

17. The composition as claimed in claim 16, in which the silyl polymer is chosen from:
a silyl polymer of general formula [Chem 2]
(R⁵O)₃₋ₚ(R⁴)ₚSi—R⁶—O—R²—O—R⁶—Si(R⁴)ₚ(OR⁵)₃₋ₚ
a silyl polymer of general formula 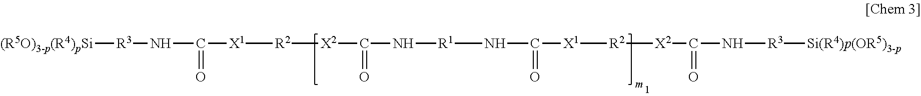
a silyl polymer of general formula 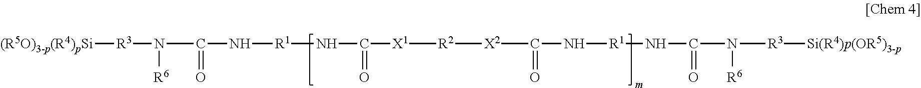
a silyl polymer of general formula 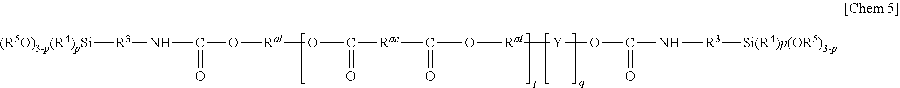
in which:
X¹ and X² represent, independently of each other, an oxygen atom or an —NH— group;
R¹ represents a divalent hydrocarbon-based radical comprising from 5 to 15 carbon atoms, which may be aromatic or aliphatic, linear, branched or cyclic;
R⁰ represents a linear or branched divalent alkylene radical comprising from 3 to 6 carbon atoms;
R³ represents a linear or branched divalent alkylene radical comprising from 1 to 6 carbon atoms, R³ preferably representing methylene or n-propylene;
R² represents a polyether block —R^{pe}—[OR^{pe}]ₙ— in which R^{pe} represents a linear or branched divalent alkylene radical comprising from 2 to 4 carbon atoms;
R⁴ and R⁵, which may be identical or different, each represent a linear or branched alkyl radical comprising from 1 to 4 carbon atoms;
R⁶ represents a hydrogen atom, a phenyl radical, a linear, branched or cyclic alkyl radical comprising from 1 to 6 carbon atoms, or a 2-succinate radical of formula 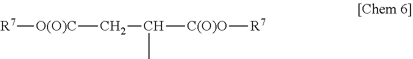
in which R⁷ is a linear or branched alkyl radical comprising from 1 to 6 carbon atoms;
n is an integer such that the number-average molecular mass of the polyether block —[OR^{pe}]ₙ— ranges from 300 g/mol to 40 000 g/mol in the polymers of general formulae [Chem 2], [Chem 3] and [Chem 4];
m₁ is zero or an integer;
m₁ is such that the number-average molecular mass of the polymer of general formula [Chem 3] ranges from 500 g/mol to 50 000 g/mol;
m is an integer other than zero;
m is such that the number-average molecular mass of the polymer of general formula [Chem 4] ranges from 500 g/mol to 50 000 g/mol;
p is an integer equal to 0, 1 or 2;
R^{al} represents a divalent hydrocarbon-based radical derived from a diol by replacement of each of the two hydroxyl groups with a free valency or represents the radical R²;
R^{ac} represents a divalent hydrocarbon-based radical derived from a dicarboxylic acid by replacement of each of the two carboxyl groups COOH with a free valency;
[Y]_{q} represents a repeating unit of general formula 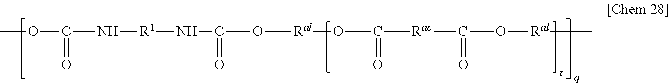
t is a number such that the polyester diol of general formula 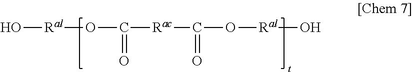
has a hydroxyl number IOH of between 4 and 60 mg KOH/g;
q is an integer other than zero;
t and q are such that the number-average molecular mass of the polymer of general formula [Chem 5] is between 400 g/mol and 50 000 g/mol.

18. The composition as claimed in claim 16, in which the curing catalyst is selected from the group consisting of amines, organometallic compounds, acids and derivatives thereof, and mixtures thereof.

19. The composition as claimed in claim 16, also comprising a tackifying resin.

20. The composition as claimed in claim 16, in which the polyvinyl ether compound comprises units derived from monomers chosen from methyl vinyl ether, ethyl vinyl ether, butyl vinyl ether, isobutyl vinyl ether, isopropyl vinyl ether, propyl vinyl ether, octyl vinyl ether or combinations thereof.

21. The composition as claimed in claim 16, in which the polyvinyl ether compound is selected from the group consisting of poly(methyl vinyl ether), poly(ethyl vinyl ether), poly(butyl vinyl ether), poly(isobutyl vinyl ether), poly(isopropyl vinyl ether), poly(propyl vinyl ether), poly(octyl vinyl ether), copolymers thereof and mixtures thereof.

22. The composition as claimed in claim 16, in which the polyvinyl ether compound has a K value of from 40 to 120.

23. The composition as claimed in claim 16, in which the polyvinyl ether compound has a glass transition temperature of from −60 to 0° C.

24. The composition as claimed in claim 16, in which the polyvinyl ether compound has a content of from 1% to 60% by weight, relative to the total weight of the composition.

25. The composition as claimed in claim 16, also comprising at least one silsesquioxane.

26. The composition as claimed in claim 16, said composition being a one-component composition.

27. The composition as claimed in claim 16, said composition being a two-component composition comprising:
a part A comprising said at least one silyl polymer; and
a part B comprising said at least one curing catalyst;
said at least one polyvinyl ether compound being present in part A and/or in part B of the composition.

28. An adhesive comprising the composition as claimed in claim 16.

29. A self-adhesive article comprising at least one support layer and at least one layer of the composition as claimed in claim 16.

30. The self-adhesive article as claimed in claim 29, said article being chosen from dressings, bandages and medical tapes.

31. The composition as claimed in claim 19, wherein the tackifying resin is selected from the group consisting of terpene phenolic resins, hydrocarbon resins, rosin resins; acrylic resins and mixtures thereof.
